(19) United States
(12) Patent Application Publication
(10) Pub. No.: US 2026/0076894 A1
(43) Pub. Date: Mar. 19, 2026

(54) ORAL CARE COMPOSITIONS COMPRISING HOPS AND TIN

(71) Applicant: The Procter & Gamble Company, Cincinnati, OH (US)

(72) Inventors: Steven Hamilton HOKE, II, West Chester, OH (US); George Kavin MORGAN, III, Hamilton, OH (US); Andrew Thomas SCHOLLE, Madeira, OH (US); David C. BOM, Union, KY (US); Hongyang LI, Mason, OH (US)

(21) Appl. No.: 19/325,563

(22) Filed: Sep. 11, 2025

Related U.S. Application Data
(60) Provisional application No. 63/694,328, filed on Sep. 13, 2024.

(51) Int. Cl. A61K 8/9789; A61K 8/21; A61K 8/27; A61K 8/365; A61K 8/46; A61Q 11/00

(52) U.S. Cl. CPC A61K 8/9789; A61K 8/21; A61K 8/27; A61K 8/365; A61K 8/46; A61Q 11/00

(57) ABSTRACT

An oral care composition is provided and includes hops acid, a tin ion source, a sulfur source, and a soluble metal salt that prevents the formation of methyl mercaptan. The soluble metal salt may include a copper salt, a silver salt, a zinc salt, or combinations thereof. The hops acid may be from a hops extract. The sulfur source may be an impurity, and the soluble metal salt may prevent the formation of unacceptable off-notes, which may be produced in the composition under reducing conditions.

### CROSS REFERENCE TO RELATED APPLICATION

This application claims the benefit of U.S. Provisional Application No. 63/694,328, filed Sep. 13, 2024.

### FIELD

The disclosure relates generally to oral care compositions comprising hops and, more particularly, relates to oral care compositions comprising hops and tin with an improved sensorial experience.

### BACKGROUND

Oral care compositions, such as toothpaste and/or dentifrice compositions, can be applied to the oral cavity to clean and/or maintain the aesthetics and/or health of the teeth, gums, and/or tongue. Additionally, many oral care compositions are used to deliver active ingredients directly to oral care surfaces. Natural compounds with antibacterial activity, such as hops, can be incorporated into oral care compositions to provide antibacterial and/or anticavity activity. Natural antibacterial agents, such as hops, can include mixtures of compounds, oils, flavonoids, and/or other flavor compounds. The use of hops beta acid extract in oral care compositions has the potential to deliver enhanced oral care benefits. Incorporating natural compounds into oral care compositions may affect the shelf-stability of the compositions. There is a need for oral care compositions with improved shelf-stability.

### SUMMARY

In an embodiment, an oral care composition is provided and includes a hops extract, where the hops extract comprises naturally occurring dimethyl disulfide and hops acids, a tin ion source, and a soluble metal ion source that prevents the accumulation of methyl mercaptan in the composition. The soluble metal ion source may include a metal salt, a metal oxide, or a combination thereof. The soluble metal ion source may include a copper ion source, a silver ion source, a zinc ion source, or combinations thereof.

In an embodiment, an oral care composition is provided and includes hops acids, and a naturally occurring sulfur source, such as dimethyl disulfide, a tin ion source, and a soluble metal ion source that prevents the accumulation of methyl mercaptan in the composition. The soluble metal ion source may include a metal salt, a metal oxide, or a combination thereof. The soluble metal ion source may include a copper ion source, a silver ion source, a zinc ion source, or combinations thereof.

### DETAILED DESCRIPTION

The present disclosure is directed towards oral care compositions comprising a reducing environment (e.g., due to the presence of stannous ions) in combination with a hops extract, which contains a residual sulfur source, and a soluble metal ion source that reduces the formation of methyl mercaptan (MM) in the composition throughout its shelf life.

Hops extracts and the components contained therein have been associated with a range of health benefits including antibacterial activity against oral pathogens. Combining hops extracts with other oral care active ingredients or stabilizers, like stannous fluoride or stannous chloride, may enhance the efficacy of an oral care composition by combining the activity of hops extract with the activity of stannous. Such a composition may have enhanced anti-gingivitis, anti-caries, or anti-malodor efficacy. However, a hops extract or oil is a natural source of dimethylsulfoxide and dimethyldisulfide (DMDS), which can break down under proper reducing conditions to generate methyl mercaptan. DMDS is difficult and expensive to remove completely by physical processing prior to incorporation into an oral care composition. Stannous produces a reducing environment in toothpastes that, in the presence of hops extract with residual DMDS, can interact to produce methyl mercaptan (MM).

To humans, methyl mercaptan is perceived as a sulfurous, rotten eggs smell that is perceptible at sub-ppm levels. Methyl mercaptan has a low boiling point (i.e., about 5.95° C.) and a strong, undesirable odor being one of the molecules leading to bad breath. Reducing the amount of volatile thiols in an oral care product is desirable.

As such, there is a need for oral care compositions with hops extract that produces less methyl mercaptan under reducing conditions. There is an additional need for a combination of stannous and hops with residual DMDS in a toothpaste with reduced accumulation of MM in those dentifrice formulations, while delivering a delightful, enjoyable, and refreshing experience.

Copper salts like copper gluconate (527-09-3) and copper chlorophyllin (11006-34-1) have been used in oral care and confectionary products. For example, copper gluconate, sodium copper chlorophyllin or a copper salt have been used to maintain fresh breath in dentifrice, mouth rinses, confectionary products and pet treats. Copper salts like copper gluconate function as a breath freshener because it sequesters the MM released by oral bacteria. However, it is believed that copper salts have not been used in conjunction with a hop-containing oral care composition to prevent the buildup of MM in the container.

A proposed mechanism for the sequestration of thiol-containing molecules by copper salts is described by Turbeville et al., The Chemistry of Copper-Containing Sulfur Adsorbents in the Presence of Mercaptans, Cat. Today, 2006, pp. 519-525. Briefly, the addition of Cu(II) to a thiol-containing solution leads to a rapid reduction of Cu(II) and the formation of a Cu(I)-thiol complex. The mechanism of Cu(II) reduction and Cu(I) complex formation as well as the kinetics of Cu(I) oxidation strongly depend on the structural properties of the individual thiols investigated. U.S. Patent Application No. 2006/0286207 describes the use of copper gluconate to prevent “skunking” in beer or other hops containing beverages. The copper gluconate is added to the beverage preventing the breakdown of the DMDS into MM. Similarly, International Patent Application Publication No. WO2024/112613A1 describes the use of copper gluconate to prevent the formation of MM and subsequent “skunking” in beer and cannabis containing beverages. Oral care compositions are very different from beer or a cannabis beverage. For example, a hops-containing toothpaste is a thick medium that can contain oral care active ingredients, such as a fluoride source, and experiential ingredients including flavor. Similarly, mouthwash may contain significantly different ingredients relative to beer or a cannabis beverage. It is not believed that copper gluconate or an applicable Cu²⁺ salt or mixture of copper salts would be effective at inhibiting MM in a hops-containing toothpaste or mouthwash merely because it worked in a beer or malt application.

While not wishing to be bound by theory, it is believed that the combination of stannous, hops extract with residual DMDS, and certain metal salts like those of copper, zinc, or silver is effective to reduce the formation of MM in an oral care composition. If the metal salt comprises copper, suitable copper salts that can reduce the amount of MM in an oral care composition can include, but are not limited to, copper gluconate, copper sulfate, copper lactate, sodium or potassium copper chlorophyllin, copper sulfate, copper acetate or mixtures thereof. The metal salt or oxide should have sufficient solubility to be effectively incorporated into the oral care composition to be effective. The type and amount of metal salt or oxide may be chosen to reduce any negative side effects like intense color change or undesired taste of the composition.

Importantly, it has been found that some copper salts work well while others do not function well, are less efficient, or cause coloration issues that are difficult to overcome. Copper chlorophyllin, for example, is less efficient than lower molecular weight Cu²⁺ salts and produces an intense dark green color, which may be undesirable. Other potentially useful copper salts include, but aren't limited to, copper oxide, copper lactate and copper acetate, because their lower molecular weight would allow effective use levels without as much influence from their inherent color. Another copper salt includes copper citrate, though the limited water solubility of copper citrate may present a challenge to incorporate in aqueous applications like an alcohol-free mouth rinse or high-water toothpaste application.

In further embodiments, the oral care composition may be passed over Cu⁰ metal where the copper takes the form of one or more beads, rods, foils, or powders. However, direct use of copper solids in the oral care composition may be inefficient, requiring a large excess of copper in the composition leading to undesirable side effects with respect to taste, color, exposure, or impact on other ingredients like fluoride. It is also possible that a small amount of copper fluoride (CuF₂) could be used complementing the functionality of stannous fluoride (SnF₂) while simultaneously controlling the generation of methyl mercaptan.

Additionally, without wishing to be bound by theory, oral care compositions comprising stannous ions are believed to produce low oxygen activity compositions when in a sealed, low-oxygen-permeability package (e.g., toothpaste tube or mouthwash bottle) because stannous ions are oxidized to stannic ions consuming residual oxygen inside of the package. When oxygen is dissolved in water, its equilibrium concentration is about 1.23Í10⁻³ M at 25° C. and 1 atm. It most probably reacts with stannous ion as shown in the following equation:

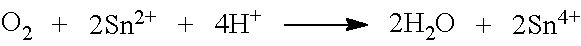

The concentration of stannous ion required for complete reaction with oxygen in a saturated solution is about 2.46Í10⁻³ M. This is approximately 300 ppm Sn(II) ion in water. By reducing the oxygen activity, the hops stability is increased in the presence of stannous by reducing the formation peroxides, prenyl radicals, and prenyl thiols. Thus, the challenge in such compositions is primarily the prevention of accumulating MM in the oral care composition in the presence of both Sn and DMDS with less need to manage the formation of prenyl thiols.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

“About” modifies a particular value by referring to a range of plus or minus 20% or less of the stated value (e.g., plus or minus 15% or less, 10% or less, or even 5% or less).

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, toothpaste, tooth gel, subgingival gel, emulsion, mouth rinse, mousse, foam, mouth spray, lozenge, chewable tablet, chewing gum, tooth whitening strips, floss and floss coatings, breath freshening dissolvable strips, unit-dose composition, fibrous composition, or denture care or adhesive product. The oral care composition may also be incorporated onto strips or films for direct application or attachment to oral surfaces, such as tooth whitening strips. Examples of emulsion compositions include the emulsions compositions of U.S. Pat. No. 11,147,753, jammed emulsions, such as the jammed oil-in-water emulsions of U.S. Pat. No. 11,096,874. Examples of unit-dose compositions include the unit-dose compositions of U.S. Patent Application Publication No. 2019/0343732.

The term “dentifrice composition”, as used herein, includes tooth or subgingival paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients useful in embodiments of the present invention can include the usual and conventional components of mouthwashes or mouth rinses. Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health, e.g., toothpaste, mouth rinse, floss, toothpicks, spray, water irrigator, massager.

The term “total water content” as used herein means both free water and water that is bound by other ingredients in the oral care composition.

For the purpose of this description, the relevant molecular weight (MW) to be used is that of the material added when preparing the composition, e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at 23° C. (i.e., room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in relation to embodiments of the present invention. When a range of any type is disclosed or claimed, the intent is to disclose or claim individually each possible number that such a range could reasonably encompass, including end points of the range as well as any sub-ranges and combinations of sub-ranges encompassed therein.

The oral care composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care composition can include a variety of active and inactive ingredients, such as, for example, but not limited to a hops extract, a dicarboxylic acid, a tin ion source, a calcium ion source, water, a fluoride ion source, zinc ion source, one or more polyphosphates, humectants, surfactants, other ingredients, and the like, as well as any combination thereof, as described below. The section headers below are provided for organization and convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

### Sulfur Source

Oral care compositions of the present invention can comprise a sulfur source. The sulfur source may be a residual sulfur source, which refers to a sulfur source that is present in the composition but is not deliberately added to the composition. It is meant to include compositions whereby the sulfur source is present only as an impurity of one of the other materials deliberately added (e.g., a natural extract) or that entered the composition during the manufacturing process. The sulfur source may include, for example, dimethylsulfide, dimethylsulfoxide, dimethyldisulfide (DMDS), dimethyltrisulfide (DMTS), methyl mercaptan, prenyl mercaptan, or combinations thereof.

In various embodiments, the composition may include a residual sulfur source in a range of from greater than 0 ppm to 500 ppm, 1 ppm to 200 ppm, 1 ppm to 100 ppm, or 1 ppm to 50 ppm. For example, the composition may include greater than 0 ppm to 500 ppm, 1 ppm to 200 ppm, 1 ppm to 100 ppm, or 1 ppm to 50 ppm of DMDS.

### Soluble Metal Ion Source

Oral care compositions of the present invention can comprise a soluble metal ion source, such as a metal salt or metal oxide, that helps prevent the formation of off-odors. A soluble metal salt or metal oxide may help prevent the formation of methyl mercaptan due to the interaction of a reducing agent, such as tin, and the sulfur source. The soluble metal salt or metal oxide may comprise a copper salt, a copper oxide, a silver salt, a silver oxide, a zinc salt, a zinc oxide, or combinations thereof. For example, in various embodiments, the soluble metal ion source may be a single copper salt or a mixture of copper salts. In other applications, it may be desirable to blend the copper salt with one or more other salts including, but not limited to, a zinc salt, a silver salt, or a combination thereof. The soluble metal salt may include, for example, copper gluconate, sodium copper chlorophyllin, potassium copper chlorophyllin, copper sulfate, copper citrate, copper lactate, copper (I) acetate, copper (II) acetate, copper oxide, silver nitrate, silver oxide, zinc gluconate, zinc oxide, zinc lactate, zinc citrate, or combinations thereof. The metal ion source can include a polymer bound metal salt, such as a polymer bound copper II salt. The soluble metal salt or metal oxide may be substantially free of, essentially free of, or free of a zinc salt or zinc oxide.

The soluble metal salt may include a copper salt, such as a Cu¹⁺ or Cu²⁺ compound, a Cu⁰ metal. The soluble metal salt may include, for example, a Cu¹⁺ salt that oxidizes in the composition to generate a Cu²⁺ salt.

The oral care composition can comprise 0.01% to 15%, 0.1% to 10%, 0.5% to 5%, 1 to 20%, or 10% or less, by weight of the oral care composition, of a soluble metal salt or metal oxide.

The oral care composition can comprise from greater than 0 ppm to 10,000 ppm, greater than 0 ppm to 8,000 ppm, greater than 0 ppm to 5,000 ppm, greater than 0 ppm to 2,000 ppm, greater than 0 to 500 ppm, greater than 0 to 100 ppm, greater than 0 to 50 ppm, greater than 0 to 20 ppm, or greater than 0 to 10 ppm, by weight of the oral care composition, of a soluble metal salt or metal oxide.

The oral care composition can comprise from greater than 0 ppm to 1,500 ppm, greater than 0 ppm to 1,000 ppm, greater than 0 ppm to 500 ppm, greater than 0 ppm to 200 ppm, greater than 0 to 100 ppm, greater than 0 to 50 ppm, greater than 0 to 25 ppm, greater than 0 to 20 ppm, or greater than 0 to 10 ppm, by weight of the oral care composition, of a metal ion from a soluble metal salt or metal oxide.

In an embodiment, the metal ion is added via the soluble metal salt or metal oxide at a level that does not noticeably affect the smell, taste, or appearance of the oral care composition separate from the effect on methyl mercaptan formation. In an embodiment, the metal ion is added at a level that has a minimally negative impact on the smell, taste, or appearance of the oral care composition, in addition to the desired effect to reduce the sulfurous odor by preventing the formation of methyl mercaptan. For example, less than 25 ppm of copper gluconate, such as 22 ppm, may provide sufficient efficacy without adding negative off-notes.

The addition of a soluble metal salt or metal oxide as described herein to an oral care composition comprising a sulfur source and a reducing agent, such as tin, results in a reduced methyl mercaptan peak area. Compared to the composition without the soluble metal salt or metal oxide, the reduction may be, for example, 10% to 100%, 25% to 100%, 40% to 100%, 50% to about 100%, 75% to 100%, or about 85% to 100%.

Humulus lupulus

Oral care compositions of the present invention can comprise hops. The hops can comprise at least one hops compound from Formula I and/or Formula IV. The compound from Formula I and/or Formula IV can be provided by any suitable source, such as an extract from Humulus lupulus or Hops, Humulus lupulus itself, a synthetically derived compound, and/or salts, prodrugs, or other analogs thereof. The hops extract can comprise one or more hops alpha acids, one or more hops iso-alpha acids, one or more hops beta acids, one or more hops oils, one or more flavonoids, one or more solvents, and/or water. Suitable hops alpha acids (generically shown in Formula I) can include humulone (Formula II), adhumulone, cohumulone, posthumulone, prehumulone, and/or mixtures thereof. Suitable hops iso-alpha acids can include cis-isohumulone and/or trans-isohumulone. The isomerization of humulone into trans-isohumulone can be represented by Formula III.

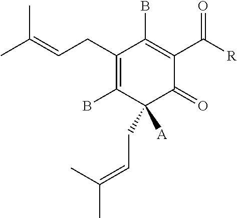

Formula I. Hops Alpha Acids. A is the acidic hydroxyl functional group in the alpha position, B are the acidic hydroxyl functional groups in the beta position, and R is an alkyl functional group.

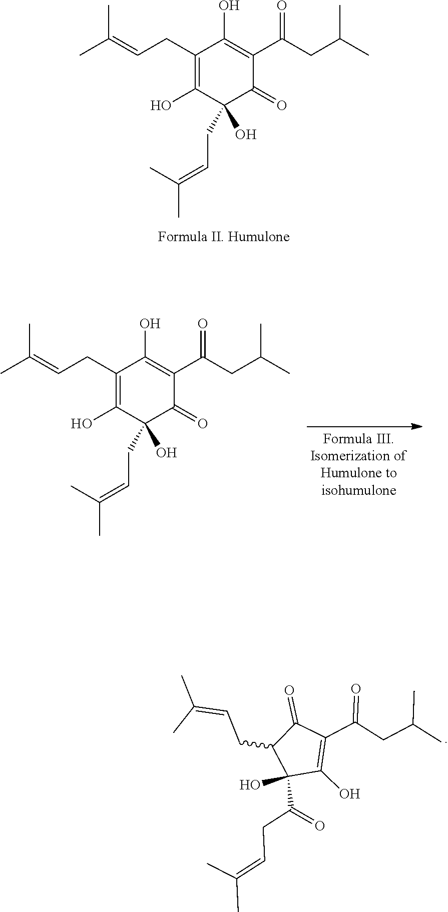

Suitable hops beta acids can include lupulone, adlupulone, colupulone, and/or mixtures thereof. A suitable hops beta acid can include a compound as described in Formula IV, V, VI, and/or VII.

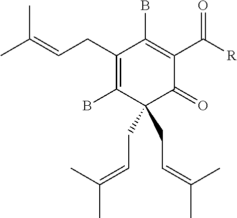

Formula IV. Hops Beta Acids. B are the acidic hydroxyl functional groups in the beta position and R is an alkyl functional group.

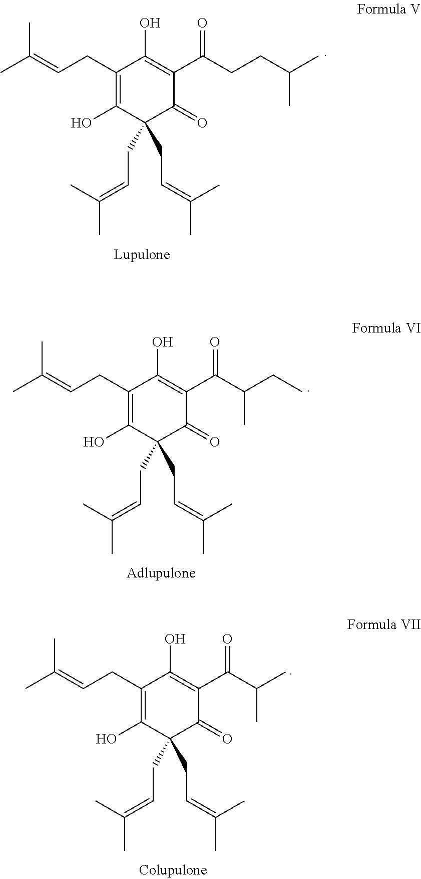

While hops alpha acids can demonstrate some antibacterial activity, hops alpha acids also have a bitter taste. The bitterness provided by hops alpha acids can be suitable for beer, but they are not suitable for use in oral care compositions. In contrast, hops beta acids can be associated with a higher antibacterial and/or anticaries activity, but not as bitter a taste. Thus, a hops extract with a higher proportion of beta acids to alpha acids than normally found in nature, can be suitable for use in oral care compositions for use as an antibacterial and/or anticaries agent. The oral care composition may be unfermented.

A natural hops source can comprise 2% to 12%, by weight of the hops source, of hops beta acids depending on the variety of hops. Hops extracts used in other contexts, such as in the brewing of beer, can comprise 15% to 35%, by weight of the extract, of hops beta acids. The hops extract desired herein can comprise at least 35%, at least 40%, at least 45%, 35% to 95%, 40% to 90%, or 45% to 99%, of hops beta acids. The hops beta acids can be in an acidic form (i.e., with attached hydrogen atom(s) to the hydroxyl functional group(s)) or as a salt form.

A suitable hops extract is described in detail in U.S. Pat. No. 7,910,140, which is herein incorporated by reference in its entirety. The hops beta acids desired can be non-hydrogenated, partially hydrogenated by a non-naturally occurring chemical reaction, or hydrogenated by a non-naturally occurring chemical reaction. The hops beta acid can be essentially free of or substantially free of hydrogenated hops beta acid and/or hops acid. A non-naturally occurring chemical reaction is a chemical reaction that was conducted with the aid of chemical compound not found within Humulus lupulus, such as a chemical hydrogenation reaction conducted with high heat not normally experienced by Humulus lupulus in the wild and/or a metal catalyst.

A natural hops source can comprise 2% to 12%, by weight of the hops source, of hops alpha acids. Hops extracts used in other contexts, such as in the brewing of beer, can comprise 15% to 35%, by weight of the extract, of hops alpha acids. The hops extract desired herein can comprise less than 10%, less than 5%, less than 1%, or less than 0.5%, by weight of the extract, of hops alpha acids.

Hops oils can include terpene hydrocarbons, such as myrcene, humulene, caryophyllene, and/or mixtures thereof. The hops extract desired herein can comprise less than 5%, less than 2.5%, or less than 2%, by weight of the extract, of one or more hops oils.

Flavonoids present in the hops extract can include xanthohumol, 8-prenylnaringenin, isoxanthohumol, and/or mixtures thereof. The hops extract can be substantially free of, essentially free of, free of, or have less than 250 ppm, less than 150 ppm, and/or less than 100 ppm of one or more flavonoids.

The hops extract can be substantially free of, essentially free of, free of dimethyl disulfide (DMDS). The hops extract may contain less than 500 ppm, less than 250 ppm, less than 150 ppm, less than 100 ppm, and/or less than 50 ppm of dimethyl disulfide (DMDS). The hops extract may contain more than 0.1 ppm, more than 1 ppm, or more than 10 ppm of DMDS.

The hops extract can be substantially free of, essentially free of, or free of ocimene. The hops extract may contain residual ocimene.

As described in U.S. Pat. No. 5,370,863, hops acids have been previously added to oral care compositions. However, the oral care compositions taught by U.S. Pat. No. 5,370,863 only included up to 0.01%, by weight of the oral care composition. While not wishing to be bound by theory, it is believed that U.S. Pat. No. 5,370,863 could only incorporate a low amount of hops acids because of the bitterness of hops alpha acids. A hops extract with a low level of hops alpha acids would not have this concern.

The hops compound can be combined with or free from an extract or oil from another plant, such as a species from genus Magnolia, Garcinia mangostana L., or Zizyphus joazeiro. The oral care composition may comprise less than 0.5%, less than 0.1%, or less than 0.01% of an extract from a plant other than hops, such as a species from genus Magnolia, Garcinia mangostana L., or Zizyphus joazeiro. The hops compounds can be combined with or free from a nonionic halogenated diphenyl ether, such as triclosan.

The oral care composition can comprise 0.01% to 10%, greater than 0.01% to 10%, 0.05%, to 10%, 0.1% to 10%, 0.2% to 10%, 0.2% to 10%, 0.2% to 5%, 0.25% to 2%, 0.05% to 2%, or greater than 0.25% to 2%, of hops, such as hops beta acid, as described herein. The hops, such as the hops beta acid, can be provided by a suitable hops extract, the hops plant itself, or a synthetically derived compound. The hops, such as hops beta acid, can be provided as neutral, acidic compounds, and/or as salts with a suitable counter ion, such as sodium, potassium, ammonia, or any other suitable counter ion.

The hops can be provided by a hops extract, such as an extract from Humulus lupulus with at least 35%, by weight of the extract, of hops beta acid and less than 1%, by weight of the hops extract, of hops alpha acid. The oral care composition can comprise 0.01% to 10%, greater than 0.01% to 10%, 0.05%, to 10%, 0.1% to 10%, 0.2% to 10%, 0.2% to 10%, 0.2% to 5%, 0.25% to 2%, 0.05% to 2%, or greater than 0.25% to 2%, of hops extract, as described herein.

### Dicarboxylic Acid

The oral care composition can comprise dicarboxylic acid. The dicarboxylic acid comprises a compound with two carboxylic acid functional groups. The dicarboxylic acid can comprise a compound or salt thereof defined by Formula VIII-A, Formula VIII-B, and/or Formula VIII-C.

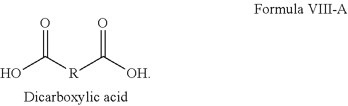

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

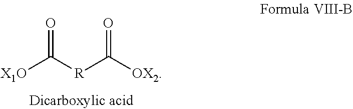

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

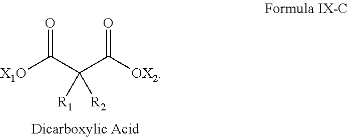

R₁ can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

The dicarboxylic acid can be added to a formulation as a neutral acid (as shown in Formula VIII-A) or as a dicarboxylate monosalt (where one of the carboxylic acid functional groups is a salt and the other is neutral), a dicarboxylate disalt (where both of the carboxylic acid functional groups are salts), or combinations thereof. Additionally, as is well known to a person of ordinary skill in the art, whether or not that one or both of the carboxylic acid functional groups of the dicarboxylic acid are neutral or charged in solution, can be influenced by the pH of the solution. For example, a neutral dicarboxylic acid can be added to an aqueous solution and one or two protons from the two carboxylic acid functional groups can be removed if the pH is lower than the pKa of the carboxylic acid functional group, as shown below in Formula VIII-D.

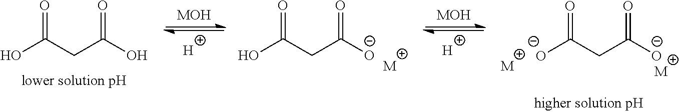

Formula VIII-D. Acid-Base Properties of Dicarboxylic Acid, wherein M is any metal.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, dihydroxymalonic acid, fumaric acid, terephthalic acid, glutaric acid, salts thereof, or combinations thereof. The dicarboxylic acid can comprise suitable salts of dicarboxylic acid, such as, for example, when the dicarboxylic acid includes a salt of oxalic acid: monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

Suitable dicarboxylic acid compounds include malonic acid, methylmalonic acid, tartronic acid, malic acid, dimethylmalonic acid, mesoxalic acid, dihydroxymalonic acid, oxalic acid, salts thereof, or combinations thereof. These dicarboxylic acid compounds are particularly suitable as these compounds have been shown to have an unexpectedly high whitening benefit. While not wishing to be bound by theory, it is believed that particular dicarboxylic acid compounds have an unexpectedly high affinity to certain cationic crosslinking agents typically found in the colored matrix on the oral hard tissue surfaces, thereby resulting in the removal of stain from the surface.

Suitable dicarboxylic acid compounds include dicarboxylic acids described by Formula VIII-A, wherein R is null, comprises a methylene or ethylene with one or two substitutions, and/or an acetyl group.

Without being bound by theory, it is hypothesized that the whitening efficacy of the dicarboxylic acids and their corresponding anions is driven by the ability of the dicarboxylic acid to reach and remove cationic bridges between chromophores and the tooth surface as well as chromophores and the pellicle proteins.

### Fluoride

The oral care composition can comprise fluoride, which can be provided by a fluoride ion source. The fluoride ion source can comprise one or more fluoride containing compounds, such as stannous fluoride, sodium fluoride, titanium fluoride, calcium fluoride, calcium phosphate silicate fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing 50 ppm to 5000 ppm, and preferably 500 ppm to 3000 ppm of free fluoride ions. To deliver the desired amount of fluoride ions, the fluoride ion source may be present in the oral care composition at an amount of 0.0025% to 5%, 0.01% to 10%, 0.2% to 1%, 0.5% to 1.5%, or 0.3% to 0.6%, by weight of the oral care composition. Alternatively, the oral care composition can comprise less than 0.1%, less than 0.01%, be essentially free of, be substantially free of, or be free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, K, Cu, Ag, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise 0.01% to 10%, 1% to 5%, or 0.5% to 15% of metal and/or a metal ion source.

### Tin

An oral care composition according to embodiments of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise 0.0025% to 5%, 0.01% to 10%, 0.2% to 1%, 0.4% to 1%, or 0.3% to 0.6%, by weight of the oral care composition, of tin and/or a tin ion source. Alternatively, the oral care composition can be essentially free of, substantially free of, or free of tin.

### Antibacterial Agents

The oral care composition can comprise one or more antibacterial agents. Suitable antibacterial agents include any molecule that provides antibacterial activity in the oral cavity. Suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof.

The oral care composition can comprise 0.01% to 10%, 1% to 5%, or 0.5% to 15% of an antibacterial agent.

### Bioactive Materials

The oral care composition can also include bioactive materials suitable for the remineralization of a tooth. Suitable bioactive materials include bioactive glasses, Novamin™, Recaldent™, hydroxyapatite, one or more amino acids, such as, for example, arginine, citrulline, glycine, lysine, or histidine, or combinations thereof. Suitable examples of compositions comprising arginine are found in U.S. Pat. Nos. 4,154,813 and 5,762,911, which are herein incorporated by reference in their entirety. Other suitable bioactive materials include any calcium phosphate compound. Other suitable bioactive materials include compounds comprising a calcium source and a phosphate source.

Amino acids are organic compounds that contain an amine functional group, a carboxyl functional group, and a side chain specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Bioactive glasses are comprising calcium and/or phosphate which can be present in a proportion that is similar to hydroxyapatite. These glasses can bond to the tissue and are biocompatible. Bioactive glasses can include a phosphopeptide, a calcium source, phosphate source, a silica source, a sodium source, and/or combinations thereof.

The oral care composition can comprise 0.01% to 20%, 0.1% to 10%, or 1% to 10% of a bioactive material by weight of the oral care composition.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of 0.01% to 10%, 0.2% to 1%, 0.4% to 1%, 0.5% to 1.5%, or 0.3% to 0.6%, by weight of the oral care composition. Alternatively, the oral care composition can be essentially free of, substantially free of, or free of zinc.

### Potassium

The oral care composition can comprise potassium, which can be provided by a potassium ion source. The potassium ion source can comprise one or more potassium containing compounds, such as potassium nitrate, potassium fluoride, potassium chloride, or combinations thereof.

The oral care composition can comprise 0.01% to 10%, 0.2% to 1%, 0.4% to 1%, or 0.3% to 0.6%, by weight of the oral care composition, of potassium and/or potassium ion source. Alternatively, the oral care composition can be essentially free of, substantially free of, or free of potassium.

### Quaternary Ammonium Compound

The oral care composition can include quaternary ammonium compound. The quaternary ammonium compounds in the compositions of embodiments of the present invention can include those in which one or two of the substitutes on the quaternary nitrogen has a carbon chain length (typically alkyl group) 8 to 20, typically 10 to 18 carbon atoms while the remaining substitutes (typically alkyl or benzyl group) have a lower number of carbon atoms, such as 1 to 7 carbon atoms, typically methyl or ethyl groups. Cetylpyridinium chloride, cetylpyridinium fluoride, tetradecylpyridinium chloride, N-tetradecyl-4-ethyl pyridinium chloride, domiphen bromide, benzalkonium chloride, benzethonium chloride, methyl benzethonium chloride, dodecyl trimethyl ammonium bromide, dodecyl dimethyl (2-phenoxyethyl) ammonium bromide, benzyl dimethoxystearyl ammonium chloride, quaternized 5-amino-1,3-bis(2-ethyl-hexyl)-5-methyl hexa hydropyrimidine, lauryl trimethylammonium chloride, cocoalkyl trimethylammonium chloride, cetyl trimethylammonium bromide, di-isobutylphenoxyethyl-dimethylbenzylammonium chloride, dodecyl trimethyl ammonium bromide, are exemplary of typical quaternary ammonium antimicrobial agents. Other compounds are bis [4-(R-amino)-1-pyridinium] alkanes as disclosed in U.S. Pat. No. 4,206,215 to Bailey. The pyridinium compounds are the preferred quaternary ammonium compounds, particularly preferred being cetylpyridinium, or tetradecylpyridinium halide salts (i.e., chloride, bromide, fluoride and iodide). Particularly preferred are cetylpyridinium chloride and fluoride salts.

The oral care composition can comprise at least 0.025%, at least 0.035%, at least 0.045% to 1.0%, 0.025% to 1%, or 0.01% to 10%, by weight of the composition, of the quaternary ammonium compound. Alternatively, the oral care composition can be essentially free of, substantially free of, or free of a quaternary ammonium compound.

pH

The pH of the oral care compositions as described herein can be 4 to 10, 7 to 10, greater than 7 to 10, greater than 8 to 10, greater than 7, greater than 7.5, greater than 8, greater than 9, 8.5 to 10, 4 to 7, 4 to 6, 4.5 to 6.5, 4.5 to 5.5, 4 to less than 5.5, 4.5 to less than 5.5, greater than 4 to less than 5, greater than 4 to 4.9, 4.9, 4 to 5.4, 4 to 5.3, 4 to 5.2, 4 to 5.1, 4 to 5, 4 to 4.9, 4 to 4.8, 4 to 4.7, or 4.8 to 5.3. The pH of a mouth rinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

If the oral care composition comprises one or more dicarboxylic acids, a preferred pH is below 7 or below 6 due to the pKa of the dicarboxylic acid. While not wishing to be bound by theory, it is believed that the dicarboxylic acid displays unique behavior when the pH is below 7 or below 6, but surfaces in the oral cavity can also be sensitive to a low pH. Additionally, at pH values above pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential for demineralization is greatly increased. Consequently, the oral care compositions comprising dicarboxylic acid, as described herein, can preferably have a pH 4 to 7, 4 to 6, 4.5 to 6.5, 4 to 5, 4 to less than 5, 4 to 4.9, or 4.5 to less than 5.5 to minimize metal hydroxide/metal oxide formation and any increased demineralization in the oral cavity.

The pH of the oral care composition, as described herein, can be measured either immediately upon mixing, or upon aging the composition by placing the oral care composition at ambient or accelerated temperature and humidity conditions, such as including measuring the pH at a temperature of 25° C., 30° C. and/or 40° C. with a 30%, 60% and/or 75% relative humidity for 28 days or longer prior to measuring the pH.

### Buffering Agents

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, citrates, borates, silicates, phosphates, imidazole, carboxylates, and mixtures thereof. Specific buffering agents include monosodium phosphate, disodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of 0.1% to 30%, 1% to 10%, or 1.5% to 3%, by weight of the present composition.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred polyphosphates include linear polyphosphates having the formula: XO(XPO₃)ₙX, wherein X is sodium, potassium, ammonium, or any other alkali metal cations and n averages 2 to 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of, essentially free of, or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris. Additional suitable polyphosphate examples include a polydentate polyphosphate (n>2) such as sodium acid pyrophosphate and sodium phytate.

The oral care composition can comprise 0.01% to 15%, 0.1% to 10%, 0.5% to 5%, 1 to 20%, or 10% or less, by weight of the oral care composition, of the polyphosphate source. Alternatively, the oral care composition can be essentially free of, substantially free of, or free of polyphosphate.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants.

Suitable anionic surfactants include, for example, the water soluble salts of alkyl sulfates having from 8 to 20 carbon atoms in the alkyl radical and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants include sarcosinates, such as sodium lauroyl sarcosinate, taurates, sodium lauryl sulfoacetate, sodium lauroyl isethionate, sodium laureth carboxylate, and sodium dodecyl benzene sulfonate. Combinations of anionic surfactants can also be employed.

Another suitable class of anionic surfactants are alkyl phosphates. The surface active organophosphate agents can have a strong affinity for enamel surface and have sufficient surface binding propensity to desorb pellicle proteins and remain affixed to enamel surfaces. Suitable examples of organophosphate compounds include mono-, di- or triesters represented by the general structure below:

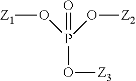

wherein Z₁, Z₂, or Z₃ may be identical or different with at least one being an organic moiety. Z₁, Z₂, or Z₃ can be selected from linear or branched, alkyl or alkenyl group of from 1 to 22 carbon atoms, optionally substituted by one or more phosphate groups; alkoxylated alkyl or alkenyl, (poly) saccharide, polyol or polyether group. Some other agents include alkyl or alkenyl phosphate esters represented by the following structure:

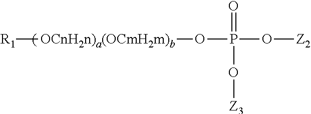

wherein R₁ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z and Z may be identical or different, each represents hydrogen, alkali metal, ammonium, protonated alkyl amine or protonated functional alkylamine, such as alkanolamine, or a R—(OCH2)(OCH)— group. Examples of suitable agents include alkyl and alkyl (poly)alkoxy phosphates such as lauryl phosphate; PPGS ceteareth-10 phosphate; laureth-1 phosphate; laureth-3 phosphate; laureth-9 phosphate; trilaureth-4 phosphate; C₁₂₋₁₈ PEG 9 phosphate; and sodium dilaureth-10 phosphate. The alkyl phosphate can be polymeric. Examples of polymeric alkyl phosphates include those containing repeating alkoxy groups as the polymeric portion, in particular 3 or more ethoxy, propoxy isopropoxy or butoxy groups.

Other suitable anionic surfactants are sarcosinates, isethionates and taurates, especially their alkali metal or ammonium salts. Examples include: lauroyl sarcosinate, myristoyl sarcosinate, palmitoyl sarcosinate, stearoyl sarcosinate oleoyl sarcosinate, or combinations thereof.

Other suitable anionic surfactants include sodium or potassium alkyl sulfates, such as sodium lauryl sulfate, acyl isethionates, acyl methyl isethionates, alkyl ether carboxylates, acyl alaninates, acyl gulatames, acyl glycinates, acyl sarconsinates, sodium methyl acyl taurates, sodium laureth sulfosuccinates, alpha olefin sulfonates, alkyl benzene sulfonates, sodium lauroyl lactylate, sodium lauryl glucosides hydroxypropyl sulfonate, and/or combinations.

A suitable taurate surfactant is represented by formula (I):

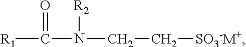

wherein R₁ is a saturated or unsaturated, straight, or branched alkyl chain with 6 to 18 C atoms; R₂ is H or methyl, and M is H, sodium, or potassium. Preferably, the R₁ is a saturated or unsaturated, straight, or branched alkyl chain with 8 to 18 C atoms. Optionally but preferably, the taurate surfactant comprises one or more selected from the group consisting of potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, and combinations thereof.

Zwitterionic or amphoteric surfactants useful herein include derivatives of aliphatic quaternary ammonium, phosphonium, and Sulfonium compounds, in which the aliphatic radicals can be straight chain or branched, and one of the aliphatic substituents contains from 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577. Typical alkyl dimethyl betaines include decyl betaine or 2-(N-decyl-N,N-dimethylammonio) acetate, coco-betaine or 2-(N-coco-N,N-dimethyl ammonio)acetate, myristyl betaine, palmityl betaine, lauryl betaine, cetyl betaine, cetyl betaine, stearyl betaine, etc. The amidobetaines can be exemplified by cocamidocthyl betaine, cocamidopropyl betaine (CAPB), and lauramidopropyl betaine. Other suitable amphoteric surfactants include betaines, sultaines, sodium laurylamphoacetates, alkylamphodiacetates, and/or combinations thereof.

Suitable cationic surfactants include, for example, derivatives of quaternary ammonium compounds having one long alkyl chain containing from 8 to 18 carbon atoms such as lauryl trimethylammonium chloride; cetyl pyridinium chloride; cetyl trimethyl-ammonium bromide; cetyl pyridinium fluoride or combinations thereof.

Suitable nonionic surfactants include, for example, compounds produced by the condensation of alkylene oxide groups (hydrophilic in nature) with an organic hydrophobic compound which may be aliphatic or alkylaromatic in nature. Examples of suitable nonionic surfactants can include the Pluronics® which are poloxamers, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and combinations of such materials. Other suitable non-ionic surfactants includes alkyl glucamides, alkyl glucosides, and/or combinations thereof.

The one or more surfactants can also include one or more natural and/or naturally derived surfactants. Natural surfactants can include surfactants that are derived from natural products and/or surfactants that are minimally or not processed. Natural surfactants can include hydrogenated, non-hydrogenated, or partially hydrogenated vegetable oils, olus oil, incarnata oil, candelilla cera, coco-caprylate, caprate, dicaprylyl ether, lauryl alcohol, myristyl myristate, dicaprylyl ether, caprylic acid, caprylic ester, octyl decanoate, octyl octanoate, undecane, tridecane, decyl oleate, oleic acid decylester, cetyl palmitate, stearic acid, palmitic acid, glyceryl stearate, hydrogenated, non-hydrogenated, or partially hydrogenated vegetable glycerides, Polyglyceryl-2 dipolyhydroxystearate, cetearyl alcohol, sucrose polystearate, glycerin, octadodecanol, hydrolyzed, partially hydrolyzed, or non-hydrolyzed vegetable protein, hydrolyzed, partially hydrolyzed, or non-hydrolyzed wheat protein hydrolysate, polyglyceryl-3 diisostearate, glyceryl oleate, myristyl alcohol, cetyl alcohol, sodium cetearyl sulfate, cetearyl alcohol, glyceryl laurate, capric triglyceride, coco-glycerides, lectithin, dicaprylyl ether, xanthan gum, sodium coco-sulfate, ammonium lauryl sulfate, sodium cocoyl sulfate, sodium cocoyl glutamate, polyalkylglucosides, such as decyl glucoside, cetearyl glucoside, cetyl stearyl polyglucoside, coco-glucoside, and lauryl glucoside, and/or combinations thereof. Natural surfactants can include any of the Natrue ingredients marketed by BASF, such as, for example, CegeSoft®, Cetiol®, Cutina®, Dehymuls®, Emulgade®, Emulgin®, Eutanol®, Gluadin®, Lameform®, LameSoft®, Lanette®, Monomuls®, Myritol®, Plantacare®, Plantaquat®, Platasil®, Rheocare®, Sulfopon®, Texapon®, and/or combinations thereof.

Other specific examples of surfactants include sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocamidopropyl betaine, lauramidopropyl betaine, palmityl betaine, sodium cocoyl glutamate, and the like. Additional surfactants desired include fatty acid salts of glutamate, alkyl glucoside, salts of taurates, betaines, caprylates, and/or mixtures thereof. The oral care composition can also be sulfate free. The oral care composition can comprise one or more surfactants each at a level 0.01% to 15%, 0.3% to 10%, or 0.3% to 2.5%, by weight of the oral care composition.

### Monodentate Ligand

The oral care composition can comprise one or more monodentate ligand(s) having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include 0.01% to 10%, 0.1% to 15%, 1% to 5%, or 0.0001 to 25%, by weight of the composition, of the monodentate ligand.

### Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e., with two functional groups), tridentate (i.e., with three functional groups), tetradentate (i.e., with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof. The polydentate ligand can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, maleic acid, malic acid, tartaric acid, phthalic acid, citric acid, phytic acid, pyrophosphate, tripolyphosphate, tetrapolyphosphate, hexametaphosphate, salts thereof, and/or combinations thereof.

The oral care composition can include 0.01% to 10%, 0.1% to 15%, 1% to 5%, or 0.0001 to 25%, by weight of the composition, of the polydentate ligand.

### Thickening Agent

The oral care composition can comprise one or more thickening agents. Thickening agents can be useful in the oral care compositions to provide a gelatinous structure that stabilizes the composition against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (sterculia gum), gum tragacanth, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carrageenans, gellan gum, locust bean gum, xanthan gum, carbomers, poloxamers, modified cellulose, and mixtures thereof. Carrageenan is a polysaccharide derived from seaweed. There are several types of carrageenan that may be distinguished by their seaweed source and/or by their degree of and position of sulfation. The thickening agent can comprise kappa carrageenans, modified kappa carrageenans, iota carrageenans, modified iota carrageenans, lambda carrageenan, and mixtures thereof. Carrageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, essentially free of, or substantially free of a copolymer of maleic anhydride and methyl vinyl ether. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise 0.01% to 15%, 0.1% to 10%, 0.2% to 5%, or 0.5% to about 2% of one or more thickening agents.

### Abrasive

The oral care composition of embodiments of the present invention can comprise an abrasive. Abrasives can be added to oral care formulations to help remove surface stains from teeth. The oral care composition can include a calcium abrasive and/or a non-calcium abrasive, such as a silica abrasive.

The oral care composition can comprise a calcium abrasive. The calcium abrasive can be any suitable abrasive compound that can provide calcium ions in an oral care composition and/or deliver calcium ions to the oral cavity when the oral care composition is applied to the oral cavity. The oral care composition can comprise 5% to 70%, 10% to 60%, 20% to 50%, 25% to 40%, or 1% to 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can comprise a non-calcium abrasive such as bentonite, silica gel (by itself, and of any structure), precipitated silica, amorphous precipitated silica (by itself, and of any structure as well), hydrated silica, perlite, titanium dioxide, calcium pyrophosphate, dicalcium phosphate dihydrate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the oral care compositions to tailor the polishing characteristics of the target dentifrice formulation. The oral care composition can comprise 5% to 70%, 10% to 50%, 10% to 60%, 20% to 50%, 25% to 40%, or 1% to 50%, by weight of the oral care composition, of the non-calcium abrasive.

Alternatively, the oral care composition can be essentially free of, substantially free of, essentially free of, or free of silica, alumina, or any other non-calcium abrasive. The oral care composition can comprise less than 5%, less than 1%, less than 0.5%, less than 0.1%, or 0% of a non-calcium abrasive, such as silica and/or alumina.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise 5% to 70%, 10% to 60%, 10% to 50%, 20% to 50%, 25% to 40%, or 1% to 50% of a silica abrasive.

Where the oral care composition comprises a dicarboxylic acid, the oral care composition can include a low level of or no abrasive as the dicarboxylic acid can provide a high enough whitening benefit that an abrasive is not necessary.

While mouth rinse compositions typically do not include abrasive, dentifrice compositions typically do include abrasive. However, the dentifrice compositions and/or toothpaste compositions of embodiments of the present invention can include a low level of or no abrasive. As such, the oral care composition or dentifrice composition can comprise less than 5%, 0.5% to 2%, or less than 2%, by weight of the composition, of abrasive. The oral care composition or dentifrice composition can also be essentially free of, substantially free of, or free of abrasive.

### Prenylated Flavonoids

The oral care composition can comprise prenylated flavonoid. Flavonoids are a group of natural substances found in a wide range of fruits, vegetables, grains, bark, roots, stems, flowers, tea, and wine. Flavonoids can have a variety of beneficial effects on health, such as antioxidative, anti-inflammatory, antimutagenic, anticarcinogenic, and antibacterial benefits. Prenylated flavonoids are flavonoids that include at least one prenyl functional group (3-methylbut-2-en-1-yl, as shown in Formula IX), which has been previously identified to facilitate attachment to cell membranes. Thus, while not wishing to being bound by theory, it is believed that the addition of a prenyl group, i.e. prenylation, to a flavonoid can increase the activity of the original flavonoid by increasing the lipophilicity of the parent molecule and improving the penetration of the prenylated molecule into the bacterial cell membrane. Increasing the lipophilicity to increase penetration into the cell membrane can be a double-edged sword because the prenylated flavonoid will tend towards insolubility at high Log P values (high lipophilicity). Log P can be an important indicator of antibacterial efficacy.

As such, the term prenylated flavonoids can include flavonoids found naturally with one or more prenyl functional groups, flavonoids with a synthetically added prenyl functional group, and/or prenylated flavonoids with additional prenyl functional groups synthetically added.

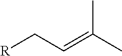

Formula IX. Prenyl Function Group with R representing the other portions of the molecule

Other suitable functionalities of the parent molecule that improve the structure-activity relationship (e.g., structure-MIC relationship) of the prenylated molecule include additional heterocycles containing nitrogen or oxygen, alkylamino chains, or alkyl chains substituted onto one or more of the aromatic rings of the parent flavonoid.

Flavonoids can have a 15-carbon skeleton with at least two phenyl rings and at least one heterocyclic ring. Some suitable flavonoid backbones can be shown in Formula X (flavone backbone), Formula XI (isoflavan backbone), and/or Formula XII (neoflavonoid backbone).

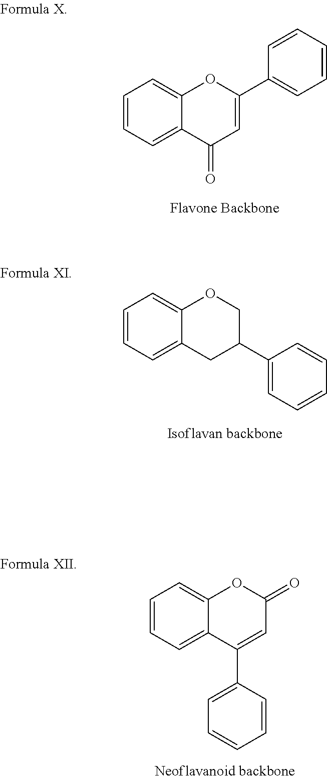 [SMILES: O=c1cc(-c2ccccc2)oc2ccccc12 ; c1ccc(C2COc3ccccc3C2)cc1 ; O=c1cc(-c2ccccc2)c2ccccc2o1]

Other suitable subgroups of flavonoids include anthocyanidins, anthoxanthins, flavanones, flavanonols, flavans, isoflavonoids, chalcones and/or combinations thereof.

Prenylated flavonoids can include naturally isolated prenylated flavonoids or naturally isolated flavonoids that are synthetically altered to add one or more prenyl functional groups through a variety of synthetic processes that would be known to a person of ordinary skill in the art of synthetic organic chemistry.

Other suitable prenylated flavonoids can include Bavachalcone, Bavachin, Bavachinin, Corylifol A, Epimedin A, Epimedin A1, Epimedin B, Epimedin C, Icariin, Icariside I, Icariside II, Icaritin, Isobavachalcone, Neobavaisoflavone, 6-Prenylnaringenin, Isoxanthohumol, 8-Prenylnaringenin, Sophoraflavanone G, (−)-Sophoranone, Xanthohumol, Quercetin, Macelignan, Kuraridin, Kurarinone, Kuwanon G, Kuwanon C, Panduratin A, 6-geranylnaringenin, Australone A, 6,8-Diprenyleriodictyol, dorsmanin C, dorsmanin F, 8-Prenylkaempferol, 7-O-Methylluteone, luteone, 6-prenylgenistein, isowighteone, lupiwighteone, and/or combinations thereof. Other suitable prenylated flavonoids include cannflavins, such as Cannflavin A, Cannflavin B, and/or Cannflavin C.

Preferably, the prenylated flavonoid has a high probability of having a MIC of less than about 25 ppm for S. aureus, a gram-positive bacterium. Suitable prenylated flavonoids include Bavachin, Bavachinin, Corylifol A, Icaritin, Isoxanthohumol, Neobavaisoflavone, 6-Prenylnaringenin, 8-Prenylnaringenin, Sophoraflavanone G, (−)-Sophoranone, Kurarinone, Kuwanon C, Panduratin A, and/or combinations thereof.

Preferably, the prenylated flavonoid has a high probability of having a MIC of less than about 25 ppm for E. coli, a gram-negative bacterium. Suitable prenylated flavonoids include Bavachinin, Isoxanthohumol, 8-Prenylnaringenin, Sophoraflavanone G, Kurarinone, Panduratin A, and/or combinations thereof.

Approximately 1000 prenylated flavonoids have been identified from plants. According to the number of prenylated flavonoids reported before, prenylated flavonones are the most common subclass and prenylated flavanols is the rarest sub-class. Even though natural prenylated flavonoids have been detected to have diversely structural characteristics, they have a narrow distribution in plants, which are different to the parent flavonoids as they are present almost in all plants. Most of prenylated flavonoids are found in the following families, including Cannabaceae, Guttiferae, Leguminosae, Moraceae, Rutaceae and Umbelliferae. Leguminosae and Moraceae, due to their consumption as fruits and vegetables, are the most frequently investigated families and many novel prenylated flavonoids have been explored. Humulus lupulus of the Cannabaceae include 8-prenylnaringenin and xanthohumol, which can play a role in the health benefits of beer.

The prenylated flavonoid can be incorporated through a hops extract, incorporated in a separately added extract, or added as a separate component of the oral care compositions disclosed herein.

Suitable prenylated flavonoids can have a particular octanol-water partitioning coefficient. The octanol-water partitioning coefficient can be used to predict the lipophilicity of a compound. Without wishing to being bound by theory, it is believed that compounds that fall within the ranges described herein will be able to enter and/or disrupt the primarily hydrophobic phospholipid bilayer that makes of the cell membrane of microorganisms. Thus, the octanol-water partitioning coefficient can be correlated to the antibacterial effect of prenylated flavonoids. Suitable prenylated flavonoids can have a log P of at least 2, at least 4, 2 to 10, 4 to 10, 4 to 7, or 4 to 7.

The oral care composition can comprise at least 0.001%, 0.001% to 5%, 0.01% to 2%, 0.0001% to 2%, or at least 0.05% of prenylated flavonoid.

### Amino Acid

The oral care composition can comprise amino acid. The amino acid can comprise one or more amino acids, peptide, and/or polypeptide, as described herein.

Amino acids, as in Formula XIII, are organic compounds that contain an amine functional group, a carboxyl functional group, and a side chain (R in Formula XIII) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula XIII, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

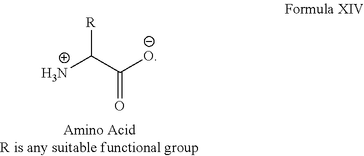

Suitable amino acids include one or more basic amino acids, one or more acidic amino acids, one or more neutral amino acids, or combinations thereof.

The oral care composition can comprise 0.01% to 20%, 0.1% to 10%, 0.5% to 6%, or 1% to 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in embodiments of the present invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, the neutral amino acids used in embodiments of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e., uncomplexed). If the neutral amino acid is in its salt form, suitable salts include salts known in the art to be pharmaceutically acceptable salts considered to be physiologically acceptable in the amounts and concentrations provided. Preferably the neutral amino acid is present in the amount of 0.0001% to 10%, preferably 0.05% to 5%, preferably 0.1% to 3%, preferably 0.5% to 3%, preferably 1% to 3%, by weight of the composition. In one aspect, the neutral amino acid is glutamine (or salt thereof). In another aspect, the neutral amino acid is proline (or salt thereof). In yet another aspect, the neutral amino acid is glycine (or salt thereof).

The oral care composition can comprise 0.0001% to 20%, 0.1% to 10%, 0.5% to 6%, or 1% to 10% of neutral amino acid, by weight of the oral care composition.

### Whitening Agent

The oral care composition may comprise 0.1% to 10%, 0.2% to 5%, 1% to 5%, or 1% to 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, or mixtures thereof.

### Humectant

The oral care composition can comprise one or more humectants, have low levels of a humectant, be essentially free of, be substantially free of, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to 70%, 5% to 50%, 10% to 60%, or 20% to 80%, by weight of the oral care composition.

### Water

The oral care composition according to embodiments of the present invention can be anhydrous, a low water formulation, or a high water formulation. In total, the oral care composition can comprise from 0% to 99%, 5% to 75%, 20% or greater, 30% or greater, 50% or greater, up to 45%, or up to 75%, by weight of the composition, of water.

In a high water oral care composition and/or toothpaste formulation, the oral care composition comprises 45% to 75%, by weight of the composition, of water. The high water oral care composition and/or toothpaste formulation can comprise 45% to 65%, 45% to 55%, or 46% to 54%, by weight of the composition, of water. The water may be added to the high water formulation and/or may come into the composition from the inclusion of other ingredients.

In a low water oral care composition and/or toothpaste formulation, the oral care composition comprises 5% to 45%, by weight of the composition, of water. The low water oral care composition can comprise 5% to 35%, 10% to 25%, or 20% to 25%, by weight of the composition, of water. The water may be added to the low water formulation and/or may come into the composition from the inclusion of other ingredients.

In an anhydrous oral care composition and/or toothpaste formulation, the oral care composition comprises less than 10%, by weight of the composition, of water. The anhydrous composition comprises less than 5%, less than 1%, or 0%, by weight of the composition, of water. The water may be added to the anhydrous formulation and/or may come into the composition from the inclusion of other ingredients.

The oral care composition can also be a mouth rinse formulation. A mouth rinse formulation can comprise 75% to 99%, 75% to 95%, or 80% to 95% of water.

The dentifrice composition can also comprise other orally acceptable carrier materials, such as alcohol, humectants, polymers, surfactants, and acceptance improving agents, such as flavoring, sweetening, coloring and/or cooling agents.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guacthol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of 0.001% to about 5%, by weight of the oral care composition. These flavoring agents generally comprise mixtures of aldehydes, ketones, esters, phenols, acids, and aliphatic, aromatic, and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide may be present at 0.25% to 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions forms include emulsion compositions, such as the emulsions compositions of U.S. Pat. No. 11,147,753, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, such as the jammed oil-in-water emulsions of U.S. Pat. No. 11,096,874, which is herein incorporated by reference in its entirety, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Methods

The oral care compositions, as described herein, can lead to oral health benefits, such as the treatment, reduction, and/or prevention of caries, cavities, gingivitis, and/or combinations thereof and/or the whitening of teeth, removing stain from teeth, and/or preventing the accumulation of stain from teeth when applied to the oral cavity. For example, a user can dispense at least a one-inch strip of a suitable oral care composition, as described herein, onto an oral care implement, such as a toothbrush, applicator, and/or tray, and applied to the oral cavity and/or teeth.

The user can be instructed to brush teeth thoroughly for at least 30 seconds, at least one minute, at least 90 seconds, or at least two minutes at least once, at least twice, or at least three times per day. The user can also be instructed to expectorate the oral care composition after the completion of the brush procedure.

The user can also be instructed to rinse with a mouthwash and/or mouth rinse composition after the completion of the brush procedure or instead of the brush procedure. The user can be instructed to swish the oral care composition thoroughly for at least 30 seconds, at least one minute, at least 90 seconds, or at least two minutes at least once, at least twice, or at least three times per day. The user can also be instructed to expectorate the oral care composition after the completion of the procedure.

The oral care compositions according to embodiments of the present invention can be used in the treatment, reduction, and/or prevention of caries, cavities, gingivitis, and/or combinations thereof. The oral care compositions according to embodiments of the present invention can be used to provide a whitening benefit, such as the whitening of teeth, removing stain from teeth, and/or preventing the accumulation of stain from teeth. For example, as described herein, hops beta acid can be useful as an antigingivitis agent. Thus, the addition of hops to any oral care composition can provide antigingivitis protection.

The oral care composition can include primary packaging, such as a tube, bottle, and/or tub. The primary package can be placed within secondary package, such as a carton, shrink wrap, or the like. Instructions for use of the oral care composition can be printed on the primary package and/or the secondary package. The scope of the method is intended to include instructions provided by a manufacturer, distributor, and/or producer of the oral care composition.

If the oral care composition is a toothpaste, the user can be instructed to dispense the toothpaste from the toothpaste tube.

The user can be instructed to apply a portion of the toothpaste onto a toothbrush. The portion of the toothpaste can be of any suitable shape, such as strip, a pea-sized amount, or various other shapes that would fit onto any mechanical and/or manual brush head. The user can be instructed to apply a strip of the toothpaste that is at least about 1 inch, at least about 0.5 inch, at least 1 inch, and/or at least 0.5 inch long to the bristles of a toothbrush, such as soft-bristled toothbrush.

The user can be instructed to apply pea-sized or grain of rice-sized portion of the toothpaste to the bristles of a toothbrush, such as in the case of use by children of less than 6 years old and/or less than 2 years old.

The user can be instructed to brush their teeth for at least about 30 seconds, at least about 1 minute, at least about 90 seconds, at least about 2 minutes, at least 30 seconds, at least 1 minute, at least 90 seconds, and/or at least 2 minutes.

The user can be instructed to brush their teeth thoroughly and/or as directed by a physician and/or dentist.

The user can be instructed to brush their teeth after each meal. The user can be instructed to brush their teeth at least once per day, at least twice per day, and/or at least three times per day. The user can be instructed to brush their teeth no more than three times a day, such as to prevent Sn staining. The user can be instructed to brush their teeth in the morning and/or in the evening prior to sleeping.

The user can be instructed to not swallow the toothpaste composition due to the inclusion of ingredients that are not suitable for ingestion, such as fluoride. However, in the case of an oral care composition comprising hops, but free of fluoride, the user may not need to be instructed to not swallow the toothpaste. The user may be instructed to expectorate (or spit out) the toothpaste composition after the cessation of the brushing cycle.

If the oral care composition is a mouth rinse, the user can be instructed to dispense the mouth rinse from a bottle containing the mouth rinse.

The user can be instructed to use the mouth rinse at least once a day, at least twice a day, and/or at least three times a day.

The user can be instructed to use the mouth rinse composition after the use of toothpaste and/or floss.

The user can be instructed to swish a portion of rinse in the oral cavity, such as between the teeth, for a period of time. The user can be instructed to vigorously swish a portion of the rinse.

The user can be instructed to use 5 mL to 50 mL, 10 mL to 40 mL, 10 mL, 20 mL, 25 mL, 30 mL, 40 mL, 2 teaspoonfuls, and/or 4 teaspoonfuls of mouth rinse.

The user can be instructed to swish the mouth rinse for at least about 30 seconds, at least about 1 minute, at least about 90 seconds, at least about 2 minutes, at least 30 seconds, at least 1 minute, at least 90 seconds, and/or at least 2 minutes.

The user can be instructed to not swallow the mouth rinse composition due to the inclusion of ingredients that are not suitable for ingestion, such as fluoride. However, in the case of an oral care composition comprising hops, but free of fluoride, the user may not need to be instructed to not swallow the mouth rinse. The user may be instructed to expectorate (or spit out) the mouth rinse composition after the cessation of the rinse cycle.

The usage instructions for the oral care composition, such as for a toothpaste composition and/or a mouth rinse composition, can vary based on age. For example, adults and children that are at least 6 or at least 2 can have one usage instruction while children under 6 or under 2 can have a second usage instruction.

The oral care composition comprising hops, as described herein, can be useful as medicament, such as in an anticavity and/or antigingivitis treatment, as described herein. Suitable medicaments include oral care compositions, toothpaste compositions, mouth rinse compositions, floss coatings, chewing gums, and/or other suitable compositions to be applied in the oral cavity.

Additionally, the oral care composition, as described herein, can be used to reduce the number and/or intensity of white spots on teeth, which can be attributable to caries presence within the oral cavity. Or the oral care composition, as described herein, can be used to reduce the redness, puffiness, tenderness, and/or swollenness of gums at the gumline immediately adjacent the surfaces of the teeth, which can be attributable to gingivitis presence within the oral cavity.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

The effects of copper, silver, and zinc salts including, but not limited to, copper gluconate, sodium copper chlorophyllin, copper citrate, copper sulfate, silver nitrate, and zinc gluconate were evaluated on the reduction of methyl mercaptan in hops extract containing toothpaste. In addition to the examples described below, an analytical approach and method was developed, which was used to evaluate the effectiveness of the copper, silver, and zinc salts on the suppression of methyl mercaptan in an oral care or other personal care product.

### Base Composition

TABLE 1 describes the hops beta acid extract provided by Hopsteiner®. Since the hops beta acids are provided as an extract, there can be some variability in the amounts of certain ingredients. However, the hops beta acid extract comprises approximately 45%, by weight of the extract, of hops beta acids and approximately 0.4%, by weight of the extract, of hops alpha acids. This is different to traditional hops extracts, which typically have more hops alpha acids than hops beta acids. Other minor ingredients may be present in the hops beta acid extract including residual sulfur. DMDS was present at a level of less than 100 ppm for the specific lots that were tested.

TABLE 1
Hops Beta Acid Extract Specification

| Ingredient | Amount (wt %) |
|---|---|
| Hops Beta Acids | 45 ± 2 |
| Hops Alpha Acids | 0.4 ± 0.3 |
| Hops oils | 1.5 ± 0.5 |
| Propylene Glycol | 20 ± 15 |
| Water | <8% |
| DMDS | <100 ppm |
| pH | 11 ± 0.5 |

The toothpaste base of TABLE 2 was prepared by combining one or more humectants, water, sweetener(s), and metal salts, buffers, dyes, and/or stabilizing agents to create a liquid mixture. The liquid mixture was homogenized at 25° C. until homogeneous and completely dissolved. Next, sodium hydroxide (50% solution) was added to the liquid mixture and the liquid mixture was homogenized at 25° C. until homogeneous and completely dissolved. A separate powder mixture was prepared by combining the abrasive silica, thickening silica, and opacifier, with any thickening agents, such as xanthan gum, carrageenan, and/or sodium carboxymethylcellulose. The powder mixture was then combined with the liquid mixture and homogenized completely. Next, the surfactant, such as sodium lauryl sulfate, flavor, and hops extract were added to the mixture. The contents were homogenized at 25° C. until homogeneous, and entrained air was removed by vacuum. As noted below, some examples described below include hops extract added in addition to that in the toothpaste base.

TABLE 2
Toothpaste Base Containing Hops Beta Acid Extract

| Ingredient | Amount (wt %) |
|---|---|
| Sorbitol | 32.135 |
| Glycerin | 26.778 |
| Water | 8.436 |
| Stannous Fluoride | 0.454 |
| Stannous Chloride (10% silica) | 0.440 |
| Sodium Gluconate | 1.025 |
| Sodium Citrate Dihydrate | 1.380 |
| Dental Silica | 15.000 |
| Xanthan Gum | 0.612 |
| Carrageenan | 1.050 |
| Sodium Lauryl Sulfate (28% Solution) | 5.170 |
| Sodium Methyl Cocoyl Taurate | 0.470 |
| Sodium Hydroxide (50% Solution) | 0.720 |
| Hops Beta Acid Extract | 0.330 |
| Composition Hole for Additional Ingredients | 6.000 |

### Metal Salt Materials

Copper Gluconate (GC 007035; Lot No. H22110909Z), Zinc Gluconate (GC 007037; Lot No. H23101884Z) and Sodium Copper Chlorophyllin (GC 012127; Lot No. H22120825Z) were provided by Archer Daniels Midland (ADM Wild). Copper Citrate (Catalog No. AAB-AA003P01) was purchased from Accela. Copper Sulfate pentahydrate (Catalog No. A11262) was purchased from Alfa Aesar. Copper(I) Acetate (Catalog No. A1540; Lot No. XOYVC-QM) was purchased from TCI. Copper(II) Acetate (Catalog No. 44355; Lot No. X29F015) was purchased from Alfa Aesar. Copper Lactate (Catalog No. ALC-FP-16039524; Lot No. A24C725N) was purchased from Alfa Chemistry. Zinc Oxide (Catalog No. 044263.30; Lot No. P021037) was purchased from Thermo Scientific. Zinc Lactate (Catalog No. QB-7424; Lot No. B35309) was purchased from Combi-Blocks. Zinc Citrate (Catalog No. QB-4752; Lot No. C80077) was purchased from Combi-Blocks. Silver Nitrate (Catalog No. 19768-0050; Lot No. B0153903) was purchased from Thermo Scientific. Copper (0) 150 mesh, 99.5% pure (CAS No. 7440-50-8; Lot No. D26Y011) was purchased from Alfa Aesar. Copper(II) Fluoride (Catalog No. 11489.09; Lot No. Y27J013) was purchased from Thermo Scientific. Silver(I) Oxide (Catalog No. 226831-5G; PCode 1003537313; Source MKCS0200) was purchased from Sigma-Aldrich. Silver(II) Oxide (Catalog No. 223638-10G; PCode 1003737505; Source MKCR6661) was purchased from Sigma-Aldrich. D.I. water was from in house Milli-Q system (Resistivity: 18.2 MΩ·cm at 25° C.).

TABLE 3 describes various metal salt stock solutions that were used in the examples below.

TABLE 3
Metal Salt Stock Solutions

| | | Copper | Sodium Copper | Copper | Copper | Silver | Zinc |
|---|---|---|---|---|---|---|---|
| | Water | Gluconate | Chlorophyllin | Sulfate | Lactate | Nitrate | Gluconate |
| Stock Solution | (wt %) | (wt %) | (wt %) | (wt %) | (wt %) | (wt %) | (wt %) |
| Copper Gluconate | 99.5 | 0.5 | — | — | — | — | — |
| Sodium Copper | 99.5 | — | 0.5 | — | — | — | — |
| Chlorophyllin | | | | | | | |
| Copper Sulfate | 99.5 | — | — | 0.5 | — | — | — |
| Copper Lactate | 99.5 | — | — | — | 0.5 | — | — |
| Silver Nitrate | 99.5 | — | — | — | — | 0.5 | — |
| Zinc Gluconate | 99.5 | — | — | — | — | — | 0.5 |
| Cu/Zn Gluconate | 99.5 | 0.25 | — | — | — | — | 0.25 |
| Blend | | | | | | | |

### Sample Preparation, Aging, and GC/MS Testing Protocol

A headspace-GC/MS approach was developed for sample preparation, analysis, identification, and quantification. The complicated toothpaste matrix made it challenging for sampling via routine extraction. A Gerstel SPME (Solid Phase Micro Extraction) Arrow headspace method was used to enrich the target (methyl mercaptan) and remove the toothpaste matrix effect. The lower extraction temperature (30° C.) was preferred for the toothpaste samples due to the high volatility of methyl mercaptan. Agilent GC/MS condition was optimized for this very volatile target on both GC and MS portion. A shorter GC ramp with a focus on the early eluting range was developed for the separation. An MS Extract Ion Chromatogram (EIC) approach was developed for the analysis of methyl mercaptan.

In some of the examples below, as specified on the respective tables, the toothpaste base was utilized along with extra hops extract (1.25 wt %). The extra hops extract was added to enhance the reaction from dimethyl disulfide to methyl mercaptan. To ensure thorough mixing, the metal salt or metal salt blend was added as a solution in water into 25 g of toothpaste base. To make 5 ppm of copper gluconate dosage, 0.025 mL of 50 mg/mL stock solution was measured by pipet and added in to 25 g of toothpaste. Then the mixture was stirred vigorously with spatula spoon until fully mixed.

To expedite the reaction from dimethyl disulfide to methyl mercaptan, the samples were aged in a hot box manufactured by Thermo Scientific at 50° C. for 2 hours. Due to its high volatility, the methyl mercaptan required more attention during sample preparation and aging. To avoid any possible transfer loss, the sample (3 g) was pre-weighed into a 25 mL SPME vial before hot box aging. The aged sample is analyzed by GC-MS within 20 minutes after removal from the hot box.

The sample needed to be cleaned up and enriched for GC volatile analysis. The complicated toothpaste matrix made it very challenging to sample via routine extraction. The static headspace solid-phase microextraction method (HS-SPME-Arrow) was applied to enrich the target (methyl mercaptan) and remove the matrix effect. All extractions were carried out using a Carbon WR/PDMS (Carboxen/Polydimethylsiloxane) PAL Smart SPME Arrow fiber (Catalog No. 100100-488-00, manufactured by Gerstel). SPME was performed on a Robotic Pro Multipurpose Sampler manufactured by Gerstel. The lower extraction temperature (30° C.) was applied for toothpaste sample due to the high volatility of methyl mercaptan.

A GC/MS Testing Protocol was developed on Agilent GC (8890)/MS (5977) equipped with an Rtx-5 MS column (Catalog No. 12626, manufactured by Restek) for the methyl mercaptan identification and quantification. With boiling point at 6° C., the methyl mercaptan required attention during sample preparation and instrument analysis. Agilent GC/MS condition was optimized for this very volatile target with focus on the early eluting portion (40° C. for 10 min). Shorter GC/MS run time (16 min) avoided the contamination of MSD by the remaining toothpaste matrix on SPME Arrow fiber. The long post run (20 min @ 280° C.) after each GC run cleaned the column residue from toothpaste base. Due to co-elution with toothpaste matrix, the Extract Ion Chromatogram (EIC) was applied for the identification and quantification of methyl mercaptan (48 m/z with R.T. @ 4.6 min). The MSD Solvent Delay needed to be set to Zero due to methyl mercaptan's early elution with Flow Rate @ 2.275 mL/min. The identification of methyl mercaptan was confirmed by its specific EIC (47 and 48 m/z), Retention Time and Mass Spectrum matching to library and standard. The EIC peak (48 m/z @ R.T. 4.6 min with Flow Rate @ 2.275 mL/min) was integrated as methyl mercaptan Peak Area with less background interference.

### Experiment 1: Copper Gluconate in Hops Toothpaste (Aged in Vial)

In Experiment 1, the effect of copper gluconate on methyl mercaptan production in a hops toothpaste was evaluated. TABLE 4 describes the composition of Examples 1A-1K. In Examples 1A-1K, the toothpaste base (25 g) was combined with hops extract (0.375 g) and copper gluconate stock solution with increasing levels of copper gluconate ranging from a 0 to 8000 ppm. For lower dosage range (<400 ppm), the desired amount of freshly prepared copper gluconate stock solution (50±1 mg in 10 mL of water) was added to the previously described 25 g toothpaste sample. For higher dosage range (>800 ppm), the desired amount of copper gluconate salt was dissolved in 2 mL of water and was added to the toothpaste base, followed by vigorous stirring with spatula spoon for 1 minute.

TABLE 4
Toothpaste Compositions

| | | | Copper | Copper | | |
|---|---|---|---|---|---|---|
| | | Additional | Gluconate | Gluconate | Copper | |
| | Toothpaste | Hops | Stock | (mg) in 2 | Gluconate | Cu metal |
| Example | Base (g) | Extract (g) | Solution (mL) | mL of water | (ppm) | (ppm) |
| 1A | 25.0 | 0.375 | 0 | — | 0 | 0 |
| 1B | 25.0 | 0.375 | 0.025 | — | 5 | 0.7 |
| 1C | 25.0 | 0.375 | 0.050 | — | 10 | 1.4 |
| 1D | 25.0 | 0.375 | 0.100 | — | 20 | 2.8 |
| 1E | 25.0 | 0.375 | 0.200 | — | 40 | 5.6 |
| 1F | 25.0 | 0.375 | 0.400 | — | 80 | 11.3 |
| 1G | 25.0 | 0.375 | 2.000 | — | 400 | 56.4 |
| 1H | 25.0 | 0.375 | — | 20 ± 1 | 800 | 112.8 |
| 1I | 25.0 | 0.375 | — | 50 ± 1 | 2000 | 281.9 |
| 1J | 25.0 | 0.375 | — | 100 ± 1 | 4000 | 563.9 |
| 1K | 25.0 | 0.375 | — | 200 ± 1 | 8000 | 1127.8 |

After mixing, 3 g of each sample was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan from sample to sample. The results based on normalized peak area, as shown in TABLE 5, demonstrate the reduction and elimination of methyl mercaptan from Examples 1B-1K, which has been compared to and normalized against the control sample (Ex. 1A) without copper gluconate.

TABLE 5
Impact of Copper Gluconate on Hops
Toothpaste in a Screw-Cap Vial

| | Copper Gluconate | Cu metal | Normalized Methyl |
|---|---|---|---|
| Example | (ppm) | (ppm) | Mercaptan Peak Area (%) |
| 1A | 0 | 0 | 100 |
| 1B | 5 | 0.7 | 63.2 |
| 1C | 10 | 1.4 | 47.1 |
| 1D | 20 | 2.8 | 37.2 |
| 1E | 40 | 5.6 | 31.8 |
| 1F | 80 | 11.3 | 24.2 |
| 1G | 400 | 56.4 | 10.5 |
| 1H | 800 | 112.8 | 2.7 |
| 1I | 2000 | 281.9 | 0 |
| 1J | 4000 | 563.9 | 0 |
| 1K | 8000 | 1127.8 | 0 |

### Experiment 2: Copper Gluconate in Hops Toothpaste (Aged in Tube)

In Experiment 2, copper gluconate functionality was tested in heat-sealed toothpaste tubes instead of the vials used in Experiment 1. TABLE 6 describes a control Example 2A and Examples 2B-2D containing copper gluconate. The Toothpaste Base (25 g) was combined with increasing amounts of the copper gluconate stock solution (50±1 mg in 10 mL of water) ranging from 0 ppm (control) to 400 ppm. The desired amount of freshly prepared copper gluconate stock solution was added to the toothpaste base, followed by vigorous stirring with spatula spoon for 1 minute.

TABLE 6
Toothpaste Compositions

| | | Copper | Copper | |
|---|---|---|---|---|
| | Toothpaste | Gluconate Stock | Gluconate | Cu Metal |
| Example | Base (g) | Solution (mL) | (ppm) | (ppm) |
| 2A | 25.0 | 0 | 0 | 0 |
| 2B | 25.0 | 0.025 | 5 | 0.7 |
| 2C | 25.0 | 0.250 | 50 | 7.0 |
| 2D | 25.0 | 2.000 | 400 | 56.4 |

After mixing, the sample was transferred into a toothpaste tube (provided by The Procter & Gamble Company) with a syringe, the tube was heat-sealed with a sealer (Model PFS-200B, No. 13BP5590270, manufactured by Impulse Sealer). Before measuring the release of methyl mercaptan, the samples were aged at 50° C. for 25 hours. When the samples were ready, 3 g of each sample was weighed into 20 mL of a Headspace sampling vial, and then the GC/MS Testing Protocol was used to measure the amount of methyl mercaptan for each sample's headspace. The results, based on the normalized peak area, as shown in TABLE 7, demonstrate the value and efficacy of copper salts on the prevention of hops derived methyl mercaptan in a stannous containing toothpaste, which has been stored in a commercial toothpaste tube. Furthermore, the results inform the appropriate usage range for copper gluconate in a commercial application.

TABLE 7
Impact of Copper Gluconate on Hops
Toothpaste in a Toothpaste Tube

| | Copper | Cu | Normalized Methyl |
|---|---|---|---|
| Example | Gluconate (ppm) | metal (ppm) | Mercaptan Peak Area (%) |
| 2A | 0 | 0 | 100 |
| 2B | 5 | 0.7 | 24.5 |
| 2C | 50 | 7.0 | 4.0 |
| 2D | 400 | 56.4 | 0.4 |

### Experiment 3: Sodium Copper Chlorophyllin in Hops Toothpaste (Aged in Vial)

In Experiment 3, the effect of copper chlorophyllin on methyl mercaptan production in a hops toothpaste was evaluated. TABLE 8 describes a control Example 3A and Examples 3B-3D containing sodium copper chlorophyllin. The toothpaste base (25 g) was combined with additional hops extract (0.375 g) and sodium copper chlorophyllin stock solution ranging from 0 ppm (control) to 4000 ppm for a total of 4 test samples. For lower dosage range (<400 ppm), the desired amount of freshly prepared copper chlorophyllin stock solution (50±1 mg in 10 mL of water) was added to the previously described 25 g toothpaste sample. For higher dosage range (>4000 ppm), the desired amount of copper chlorophyllin solution salt was dissolved in 2 mL of water and was added to the toothpaste base, followed by vigorous stirring with spatula spoon for 1 minute.

TABLE 8
Toothpaste Compositions

| | | | Sodium Copper | Sodium Copper | | |
|---|---|---|---|---|---|---|
| | | Additional | Chlorophyllin | Chlorophyllin | Sodium Copper | |
| | Toothpaste | Hops | Stock | (mg) in 2 mL | Chlorophyllin | Cu metal |
| Example | Base (g) | Extract (g) | Solution (mL) | of water | (ppm) | (ppm) |
| 3A | 25.0 | 0.375 | 0 | | 0 | 0 |
| 3B | 25.0 | 0.375 | 0.200 | | 40 | 3.5 |
| 3C | 25.0 | 0.375 | 2.000 | | 400 | 35.4 |
| 3D | 25.0 | 0.375 | | 100 ± 1 | 4000 | 353.6 |

After mixing, 3 g of each sample was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan in each sample's headspace. The results based on a normalized peak area, TABLE 9, demonstrate the reduction of methyl mercaptan in the toothpaste samples, which has been compared to and normalized against the control sample without sodium copper chlorophyllin. Unlike many other copper salts, sodium copper chlorophyllin has an intense dark green color at the levels where the sodium copper chlorophyllin was efficacious. This strong color could make the use of sodium copper chlorophyllin in a toothpaste application difficult depending on the desired color of the final composition. Additionally, the sodium copper chlorophyllin could stain teeth during use.

TABLE 9
Impact of Copper Chlorophyllin on
Hops Toothpaste in a Screw-Cap Vial

| | Sodium Copper | Cu metal | Normalized Methyl |
|---|---|---|---|
| Example | Chlorophyllin (ppm) | (ppm) | Mercaptan Peak Area (%) |
| 3A | 0 | 0 | 100 |
| 3B | 40 | 3.5 | 86.6 |
| 3C | 400 | 35.4 | 64.8 |
| 3D | 4000 | 353.6 | 6.2 |

### Experiment 4: Copper Sulfate in Hops Toothpaste (Aged in Vial)

In Experiment 4, the effect of copper sulfate on methyl mercaptan production in a hops toothpaste was evaluated. TABLE 12 describes a control Example 4A and Examples 4B and 4C containing copper sulfate. The Toothpaste Base (25 g) was combined with additional hops extract and increasing levels of copper sulfate from stock solution (50±1 mg in 10 mL of water) ranging from a 0 ppm (control) to 400 ppm for a total of 3 test samples. The desired amount of freshly prepared copper sulfate solution was added to the previously described toothpaste sample (25 g), followed by vigorous stirring with a spatula spoon for 1 minute.

TABLE 10
Toothpaste Compositions

| | | Additional | | | |
|---|---|---|---|---|---|
| | Toothpaste | Hops | Copper | Copper | Cu |
| | Base | Extract | Sulfate | Sulfate | metal |
| Example | (g) | (g) | Solution (mL) | (ppm) | (ppm) |
| 4A | 25.0 | 0.375 | 0 | 0 | 0 |
| 4B | 25.0 | 0.375 | 0.200 | 40 | 10.2 |
| 4C | 25.0 | 0.375 | 2.000 | 400 | 102.4 |

After mixing, 3 g of each sample was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan from sample to sample. The results, based on a normalized peak area, TABLE 11, demonstrate the reduction of methyl mercaptan from Examples 4B-4D, which has been compared to and normalized against the control sample (Ex. 4A) without copper sulfate.

TABLE 11
Impact of Copper Sulfate on Hops Toothpaste (Vial)

| | Copper Sulfate | Cu metal | Normalized Methyl |
|---|---|---|---|
| Example | (ppm) | (ppm) | Mercaptan Peak Area (%) |
| 4A | 0 | 0 | 100 |
| 4B | 40 | 10.2 | 6.0 |
| 4C | 400 | 102.4 | 0 |

### Experiment 5: Copper Citrate in Hops Toothpaste (Aged in Vial)

When making the copper citrate stock solution, a precipitate formed that stayed at the bottom of the flask due to its poor water solubility. Based on this observation, we formulated the copper citrate into the toothpaste to measure the impact on suppression of methyl mercaptan by two approaches. One is direct addition of copper citrate in solid powder form as Experiment 5A-5D. In this Experiment, the effect of copper citrate added in solid form on methyl mercaptan production in a hops toothpaste was evaluated. TABLE 12 describes a control Example 5A and Examples 5B-5D containing copper citrate. The Toothpaste Base (25 g) was combined with increasing levels of copper citrate ranging from a 0 ppm (control) to 180 ppm for a total of 4 test samples. The desired amount of copper citrate was added to the Toothpaste Base (25 g), followed by vigorous stirring with spatula spoon for 2 minutes to ensure the evenly mixing.

TABLE 12
Toothpaste Compositions

| | Toothpaste | Copper Citrate | Copper Citrate | Cu metal |
|---|---|---|---|---|
| Example | Base (g) | Solid (mg) | (ppm) | (ppm) |
| 5A | 25.0 | 0 | 0 | 0 |
| 5B | 25.0 | 1.4 | 56 | 18.9 |
| 5C | 25.0 | 2.6 | 104 | 35.1 |
| 5D | 25.0 | 4.5 | 180 | 60.8 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 13, demonstrate the reduction and elimination of methyl mercaptan from Examples 5B-5D, which has been compared to and normalized against the control sample (Ex. 5A) without copper citrate. This example demonstrates that some metal salts, without adequate solubility, still can be applied effectively to reduce methyl mercaptan, but it requires special handling and mixing.

TABLE 13
Impact of Copper Citrate solid on
Hops Toothpaste in a Screw-Cap Vial

| | Copper | | Normalized Methyl |
|---|---|---|---|
| | Citrate | Cu metal | Mercaptan |
| Example | (ppm) | (ppm) | Peak Area (%) |
| 5A | 0 | 0 | 100 |
| 5B | 56 | 18.9 | 12.1 |
| 5C | 104 | 35.1 | 6.7 |
| 5D | 180 | 60.8 | 4.7 |

Another way of adding copper citrate is by its saturated solution in water. In Experiment 5, the effect of copper citrate solution on methyl mercaptan production in a hops toothpaste was evaluated. TABLE 14 describes a control Example 5E and Examples 5F-5H containing copper citrate. The Toothpaste Base (25 g) was combined with 0.05 mL of copper citrate saturated solution for a total of 4 test samples. The freshly prepared over-saturated copper citrate solution (as in TABLE 14) was filtered prior to the addition to the previously described toothpaste sample (25 g), followed by vigorous stirring with a spatula spoon for 1 minute.

TABLE 14
Toothpaste Compositions

| | | Copper Citrate | Copper | |
|---|---|---|---|---|
| | Toothpaste | (mg in 10 mL | Citrate | Cu metal |
| Example | Base (g) | of water) | (ppm) | (ppm) |
| 5E | 25.0 | 0 | 0 | 0 |
| 5F | 25.0 | 500.7 | NA | NA |
| 5G | 25.0 | 1002.7 | NA | NA |
| 5H | 25.0 | 2500.4 | NA | NA |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 15, showed very limited impact on the reduction and elimination of methyl mercaptan from the Examples 5F-5H, which has been compared to and normalized against the control sample (Ex. 5E) without copper citrate. This example demonstrates that some metal salts, without adequate solubility, are difficult to use as a solution in application. Poor solubility results in poor control of methyl mercaptan if added in solution form, see Table 15.

TABLE 15
Impact of Copper Citrate solution on
Hops Toothpaste in a Screw-Cap Vial

| | Copper | | Normalized Methyl |
|---|---|---|---|
| | Citrate | Cu metal | Mercaptan |
| Example | (ppm) | (ppm) | Peak Area (%) |
| 5E | 0 | 0 | 100 |
| 5F | NA | NA | 97.7 |
| 5G | NA | NA | 98.2 |
| 5H | NA | NA | 98.3 |

### Experiment 6: Silver Nitrate in Hops Toothpaste (Aged in Vial)

In Experiment 6, the effect of silver nitrate on methyl mercaptan production in a hops toothpaste was evaluated. TABLE 16 describes a control Example 6A and Examples 6B-6D containing silver nitrate. The Toothpaste Base (25 g) was combined with increasing levels of silver nitrate stock solution (50±1 mg in 10 mL of water) ranging from a 0 ppm (control) to 20 ppm for a total of 4 test samples. The desired amount of freshly prepared silver nitrate stock solution was added to the Toothpaste Base (25 g), followed by vigorous stirring with spatula spoon for 1 minute.

TABLE 16
Toothpaste Compositions

| | | Silver Nitrate | Silver | |
|---|---|---|---|---|
| | Toothpaste | Stock | Nitrate | Ag metal |
| Example | Base (g) | Solution (mL) | (ppm) | (ppm) |
| 6A | 25.0 | 0 | 0 | 0 |
| 6B | 25.0 | 0.025 | 5 | 3.2 |
| 6C | 25.0 | 0.050 | 10 | 6.4 |
| 6D | 25.0 | 0.100 | 20 | 12.7 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 17, demonstrate the reduction and elimination of methyl mercaptan from Examples 6B-6D, which has been compared to and normalized against the control sample (Ex. 6A) without silver nitrate. This example demonstrates that other metal salts, especially silver, can be highly effective at reducing methyl mercaptan.

TABLE 17
Impact of Silver Nitrate on Hops Toothpaste in a Screw-Cap Vial

| | Silver | | Normalized Methyl |
|---|---|---|---|
| | Nitrate | Ag Metal | Mercaptan |
| Example | (ppm) | (ppm) | Peak Area (%) |
| 6A | 0 | 0 | 100 |
| 6B | 5 | 3.2 | 20.4 |
| 6C | 10 | 6.4 | 11.7 |
| 6D | 20 | 12.7 | 4.0 |

### Experiment 7: Zinc Gluconate in Hops Toothpaste (Aged in Vial)

In Experiment 7, the effect of zinc gluconate on methyl mercaptan production in a hops toothpaste was evaluated. TABLE 18 describes a control Example 7A and Examples 7B-7D containing zinc gluconate. The Toothpaste Base (25 g) was combined with increasing levels of zinc gluconate stock solution (50±1 mg in 10 mL of water) ranging from 0 ppm (control) to 20 ppm for a total of 4 test samples. The desired amount of freshly prepared zinc gluconate stock solution was added to the previously described Toothpaste Base (25 g), followed by vigorous stirring with a spatula spoon for 1 minute.

TABLE 18
Toothpaste Compositions

| | | Zinc Gluconate | Zinc | Zn |
|---|---|---|---|---|
| | Toothpaste | Stock Solution | Gluconate | metal |
| Example | Base (g) | (mL) | (ppm) | (ppm) |
| 7A | 25.0 | 0 | 0 | 0 |
| 7B | 25.0 | 0.025 | 5 | 0.7 |
| 7C | 25.0 | 0.050 | 10 | 1.4 |
| 7D | 25.0 | 0.100 | 20 | 2.9 |

Each sample (3 g) was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan in each sample's headspace. The results, based on a normalized peak area, TABLE 19, demonstrate the reduction of methyl mercaptan from Examples 7B-7D, which has been compared to and normalized against the control sample (Ex. 7A) without zinc gluconate. Compared to the efficacy observed for salts of silver and copper, zinc gluconate did not perform well.

TABLE 19
Impact of Zinc Gluconate on Hops Toothpaste (Vial)

| | Zinc | Zn | Normalized Methyl |
|---|---|---|---|
| | Gluconate | Metal | Mercaptan |
| Example | (ppm) | (ppm) | Peak Area (%) |
| 7A | 0 | 0 | 100 |
| 7B | 5 | 0.7 | 100 |
| 7C | 10 | 1.4 | 91.2 |
| 7D | 20 | 2.9 | 78.1 |

### Experiment 8: Blend of Copper Gluconate and Zinc Gluconate in Hops Toothpaste (Aged in Vial)

In Experiment 8, the effect of a blend of copper gluconate and zinc gluconate on methyl mercaptan production in a hops toothpaste was evaluated. TABLE 20 describes a control Example 7A and Examples 7B-7D containing the blend of copper gluconate and zinc gluconate. The Toothpaste Base (25 g) was combined with increasing levels of the copper gluconate/zinc gluconate blend (1:1) stock solution ranging from 0 ppm (control) to 20 ppm. The desired amount of freshly prepared copper gluconate/zinc gluconate blend stock solution (25±1 mg of copper gluconate and 25±1 mg of zinc gluconate in 10 mL of water) was added to the Toothpaste Base (25 g), followed by vigorous stirring with spatula spoon for 1 minute.

TABLE 20
Toothpaste Compositions

| | | Cu/Zn Gluconate | Zinc | Zn | Copper | Cu |
|---|---|---|---|---|---|---|
| | Toothpaste | Blend Stock | Gluconate | Metal | Gluconate | Metal |
| Example | Base (g) | Solution (mL) | (ppm) | (ppm) | (ppm) | (ppm) |
| 8A | 25.0 | 0 | 0 | 0 | 0 | 0 |
| 8B | 25.0 | 0.025 | 2.5 | 0.4 | 2.5 | 0.4 |
| 8C | 25.0 | 0.050 | 5 | 0.7 | 5 | 0.7 |
| 8D | 25.0 | 0.100 | 10 | 1.4 | 10 | 1.4 |

Each sample (3 g) was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan from sample to sample. The results, based on normalized peak area, TABLE 21, illustrate the reduction of methyl mercaptan from Examples 8B-8D, which has been compared to, and normalized against, the control sample (Ex. 8A) without copper gluconate/zinc gluconate (1:1). These results demonstrate that a combination of metal salts could be used, like, but not limited to, a copper salt in combination with a zinc salt, to control methyl mercaptan formation.

TABLE 21
Impact of Zinc Gluconate and Copper Gluconate on Hops Toothpaste (Vial)

| | | | | | | Normalized |
|---|---|---|---|---|---|---|
| | Zinc | Zn | Copper | Cu | Total | Methyl |
| | Gluconate | metal | Gluconate | metal | Metal | Mercaptan |
| Example | (ppm) | (ppm) | (ppm) | (ppm) | (ppm) | Peak Area (%) |
| 8A | 0 | 0 | 0 | 0 | 0 | 100 |
| 8B | 2.5 | 0.4 | 2.5 | 0.4 | 0.8 | 51.8 |
| 8C | 5 | 0.7 | 5 | 0.7 | 1.4 | 34.9 |
| 8D | 10 | 1.4 | 10 | 1.4 | 2.8 | 19.9 |

### Experiment 9: Blend of Copper Gluconate and Silver Nitrate in Hops Toothpaste (Aged in Vial)

In Experiment 9, the effect of a blend of copper gluconate and silver nitrate on methyl mercaptan production in a hops toothpaste was evaluated. TABLE 22 describes a control Example 9A and Examples 9B-9D containing the blend of copper gluconate and silver nitrate. The Toothpaste Base (25 g) was combined with increasing levels of the copper gluconate/silver nitrate blend (1:1) stock solution ranging from 0 ppm (control) to 20 ppm. The desired amount of freshly prepared copper gluconate/silver nitrate blend stock solution (25±1 mg of copper gluconate and 25±1 mg of silver nitrate in 10 mL of water) was added to the Toothpaste Base (25 g), followed by vigorous stirring with spatula spoon for 1 minute.

TABLE 22
Toothpaste Compositions

| | | Cu Gluconate/ | | | | |
|---|---|---|---|---|---|---|
| | | Silver Nitrate | Copper | Cu | Silver | Ag |
| | Toothpaste | Blend Stock | Gluconate | Metal | Nitrate | Metal |
| Example | Base (g) | Solution (mL) | (ppm) | (ppm) | (ppm) | (ppm) |
| 9A | 25.0 | 0 | 0 | 0 | 0 | 0 |
| 9B | 25.0 | 0.025 | 2.5 | 0.4 | 2.5 | 1.6 |
| 9C | 25.0 | 0.050 | 5 | 0.7 | 5 | 3.2 |
| 9D | 25.0 | 0.100 | 10 | 1.4 | 10 | 6.4 |

Each sample (3 g) was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan from sample to sample. The results, based on normalized peak area, TABLE 23, illustrate the reduction of methyl mercaptan from Examples 9B-9D, which has been compared to, and normalized against, the control sample (Ex. 9A) without copper gluconate/silver nitrate (1:1). These results demonstrate that a combination of metal salts could be used, like, but not limited to, a copper salt in combination with a silver salt, to control methyl mercaptan formation.

TABLE 23
Impact of Copper Gluconate and Silver Nitrate on Hops Toothpaste (Vial)

| | | | | | | Normalized |
|---|---|---|---|---|---|---|
| | Copper | Cu | Silver | Ag | Total | Methyl |
| | Gluconate | metal | Nitrate | metal | Metal | Mercaptan |
| Example | (ppm) | (ppm) | (ppm) | (ppm) | (ppm) | Peak Area (%) |
| 9A | 0 | 0 | 0 | 0 | 0 | 100 |
| 9B | 2.5 | 0.4 | 2.5 | 1.6 | 2.0 | 24.6 |
| 9C | 5 | 0.7 | 5 | 3.2 | 3.9 | 10.2 |
| 9D | 10 | 1.4 | 10 | 6.4 | 7.8 | 3.0 |

### Experiment 10: Zinc Oxide in Hops Toothpaste (Aged in Vial)

When making the zinc oxide stock solution, a precipitate formed that stayed at the bottom of the flask due to its poor water solubility. Based on this observation, zinc oxide is added directly in solid powder form as Experiment 10A-10D. In this Experiment, the effect of zinc oxide on methyl mercaptan production in a hops toothpaste was evaluated. TABLE 24 describes a control Example 10A and Examples 10B-10D containing zinc oxide. The Toothpaste Base (25 g) was combined with increasing levels of zinc oxide ranging from 0 ppm (control) to 212 ppm for a total of 4 test samples. The desired amount of zinc oxide was added to the Toothpaste Base (25 g), followed by vigorous stirring with spatula spoon for 2 minutes to ensure the evenly mixing.

TABLE 24
Toothpaste Compositions

| | | | Zinc | Zn |
|---|---|---|---|---|
| | Toothpaste | Zinc Oxide | Oxide | metal |
| Example | Base (g) | Solid (mg) | (ppm) | (ppm) |
| 10A | 25.0 | 0 | 0 | 0 |
| 10B | 25.0 | 1.3 | 52 | 41.8 |
| 10C | 25.0 | 2.6 | 104 | 83.6 |
| 10D | 25.0 | 5.3 | 212 | 170.3 |

Each sample (3 g) was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan in each sample's headspace. The results, based on a normalized peak area, TABLE 25, demonstrate the reduction of methyl mercaptan from Examples 10B-10D, which has been compared to and normalized against the control sample (Ex. 10A) without zinc oxide. Compared to the efficacy observed for salts of silver and copper, zinc oxide did not perform well.

TABLE 25
Impact of Zinc Oxide on Hops Toothpaste (Vial)

| | Zinc | Zn | Normalized Methyl |
|---|---|---|---|
| | Oxide | Metal | Mercaptan |
| Example | (ppm) | (ppm) | Peak Area (%) |
| 10A | 0 | 0 | 100 |
| 10B | 52 | 41.8 | 95.3 |
| 10C | 104 | 83.6 | 92.4 |
| 10D | 212 | 170.3 | 91.5 |

### Experiment 11: Zinc Lactate in Hops Toothpaste (Aged in Vial)

When making the zinc lactate stock solution, a precipitate formed that stayed at the bottom of the flask due to its poor water solubility. Based on this observation, zinc lactate is added directly in solid powder form as Experiment 11A-11D. In this Experiment, the effect of zinc lactate on methyl mercaptan production in a hops toothpaste was evaluated. TABLE 26 describes a control Example 11A and Examples 11B-11D containing zinc lactate. The Toothpaste Base (25 g) was combined with increasing levels of zinc lactate ranging from a 0 ppm (control) to 200 ppm for a total of 4 test samples. The desired amount of zinc lactate was added to the Toothpaste Base (25 g), followed by vigorous stirring with spatula spoon for 2 minutes to ensure the evenly mixing.

TABLE 26
Toothpaste Compositions

| | | Zinc | Zinc | Zn |
|---|---|---|---|---|
| | Toothpaste | Lactate | Lactate | metal |
| Example | Base (g) | Solid (mg) | (ppm) | (ppm) |
| 11A | 25.0 | 0 | 0 | 0 |
| 11B | 25.0 | 1.2 | 48 | 12.8 |
| 11C | 25.0 | 2.6 | 104 | 27.7 |
| 11D | 25.0 | 5.0 | 200 | 53.3 |

Each sample (3 g) was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan in each sample's headspace. The results, based on a normalized peak area, TABLE 27, demonstrate the reduction of methyl mercaptan from Examples 11B-11D, which has been compared to and normalized against the control sample (Ex. 11A) without zinc lactate. Compared to the efficacy observed for salts of silver and copper, zinc lactate did not perform well.

TABLE 27
Impact of Zinc Lactate on Hops Toothpaste (Vial)

| | Zinc | Zn | Normalized Methyl |
|---|---|---|---|
| | Lactate | Metal | Mercaptan |
| Example | (ppm) | (ppm) | Peak Area (%) |
| 11A | 0 | 0 | 100 |
| 11B | 48 | 12.8 | 96.5 |
| 11C | 104 | 27.7 | 96.0 |
| 11D | 200 | 53.3 | 97.9 |

### Experiment 12: Zinc Citrate in Hops Toothpaste (Aged in Vial)

When making the zinc citrate stock solution, a precipitate formed that stayed at the bottom of the flask due to its poor water solubility. Based on this observation, zinc citrate is added directly in solid powder form as Experiment 12A-12D. In this Experiment, the effect of zinc citrate on methyl mercaptan production in a hops toothpaste was evaluated. TABLE 28 describes a control Example 12A and Examples 12B-12D containing zinc citrate. The Toothpaste Base (25 g) was combined with increasing levels of zinc citrate ranging from 0 ppm (control) to 204 ppm for a total of 4 test samples. The desired amount of zinc citrate was added to the Toothpaste Base (25 g), followed by vigorous stirring with spatula spoon for 2 minutes to ensure the evenly mixing.

TABLE 28
Toothpaste Compositions

| | | Zinc | Zinc | Zn |
|---|---|---|---|---|
| | Toothpaste | Citrate | Citrate | metal |
| Example | Base (g) | Solid (mg) | (ppm) | (ppm) |
| 12A | 25.0 | 0 | 0 | 0 |
| 12B | 25.0 | 1.2 | 48 | 16.4 |
| 12C | 25.0 | 2.6 | 104 | 35.5 |
| 12D | 25.0 | 5.1 | 204 | 69.7 |

Each sample (3 g) was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan in each sample's headspace. The results, based on a normalized peak area, TABLE 29, demonstrate the reduction of methyl mercaptan from Examples 12B-12D, which has been compared to and normalized against the control sample (Ex. 12A) without zinc citrate. Compared to the efficacy observed for salts of silver and copper, zinc citrate did not perform well.

TABLE 29
Impact of Zinc Citrate on Hops Toothpaste (Vial)

| | Zinc | Zn | Normalized Methyl |
|---|---|---|---|
| | Citrate | Metal | Mercaptan |
| Example | (ppm) | (ppm) | Peak Area (%) |
| 12A | 0 | 0 | 100 |
| 12B | 48 | 16.4 | 95.5 |
| 12C | 104 | 35.5 | 96.9 |
| 12D | 204 | 69.7 | 96.6 |

### Experiment 13: Copper Lactate in Hops Toothpaste (Aged in Vial)

In Experiment 13, the effect of copper lactate on methyl mercaptan production in a hops toothpaste was evaluated. TABLE 30 describes a control Example 13A and Examples 13B-13D containing copper lactate. The Toothpaste Base (25 g) was combined with increasing levels of copper lactate from stock solution (50±1 mg in 10 mL of water) ranging from 0 ppm (control) to 20 ppm for a total of 4 test samples. The desired amount of freshly prepared copper lactate solution was added to the previously described toothpaste sample (25 g), followed by vigorous stirring with a spatula spoon for 1 minute.

TABLE 30
Toothpaste Compositions

| | | Copper | Copper | Cu |
|---|---|---|---|---|
| | Toothpaste | Lactate | Lactate | metal |
| Example | Base (g) | Solution (mL) | (ppm) | (ppm) |
| 13A | 25.0 | 0 | 0 | 0 |
| 13B | 25.0 | 0.025 | 5 | 1.3 |
| 13C | 25.0 | 0.050 | 10 | 2.6 |
| 13D | 25.0 | 0.100 | 20 | 5.3 |

After mixing, 3 g of each sample was weighed into a 20 mL headspace sampling vial. Before measuring the release of methyl mercaptan, the samples were aged at 50° C. for two hours. The higher temperature was utilized to accelerate aging. When the samples were ready, the GC/MS Testing Protocol was used to measure the amount of methyl mercaptan from sample to sample. The results, based on a normalized peak area, TABLE 31, demonstrate the reduction of methyl mercaptan from Examples 4B-4D, which has been compared to and normalized against the control sample (Ex. 4A) without copper lactate.

TABLE 31
Impact of Copper Lactate on Hops Toothpaste (Vial)

| | Copper | Cu | Normalized Methyl |
|---|---|---|---|
| | Lactate | metal | Mercaptan |
| Example | (ppm) | (ppm) | Peak Area (%) |
| 13A | 0 | 0 | 100 |
| 13B | 5 | 1.3 | 45.6 |
| 13C | 10 | 2.6 | 34.2 |
| 13D | 20 | 5.3 | 27.8 |

### Experiment 14: Copper (II) Acetate in Hops Toothpaste (Aged in Vial)

When making the copper (II) acetate stock solution, a precipitate formed that stayed at the bottom of the flask due to its poor water solubility. Based on this observation, copper (II) acetate is added directly in solid powder form as Experiment 14A-14D. In this Experiment, the effect of copper (II) acetate on methyl mercaptan production in a hops toothpaste was evaluated. TABLE 32 describes a control Example 14A and Examples 14B-14D containing copper (II) acetate. The Toothpaste Base (25 g) was combined with increasing levels of copper (II) acetate ranging from 0 ppm (control) to 204 ppm for a total of 4 test samples. The desired amount of copper (II) acetate was added to the Toothpaste Base (25 g), followed by vigorous stirring with spatula spoon for 2 minutes to ensure the evenly mixing.

TABLE 32
Toothpaste Compositions

| | | Copper (II) | | |
|---|---|---|---|---|
| | Toothpaste | acetate | Copper (II) | Cu metal |
| Example | Base (g) | Solid (mg) | Acetate (ppm) | (ppm) |
| 14A | 25.0 | 0 | 0 | 0 |
| 14B | 25.0 | 1.3 | 52 | 18 |
| 14C | 25.0 | 3.1 | 124 | 43 |
| 14D | 25.0 | 5.1 | 204 | 71 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 33, demonstrate the reduction and elimination of methyl mercaptan from Examples 14B-14D, which has been compared to and normalized against the control sample (Ex. 14A) without copper (II) acetate. This example demonstrates that some metal salts, without adequate solubility, still can be applied effectively to reduce methyl mercaptan, but it requires special handling and mixing.

TABLE 33
Impact of Copper (II) Acetate Solid on
Hops Toothpaste in a Screw-Cap Vial

| | Copper (II) | | Normalized Methyl |
|---|---|---|---|
| | Acetate | Cu metal | Mercaptan |
| Example | (ppm) | (ppm) | Peak Area (%) |
| 14A | 0 | 0 | 100 |
| 14B | 52 | 18 | 13.9 |
| 14C | 124 | 43 | 8.7 |
| 14D | 204 | 71 | 6.5 |

### Experiment 15: Copper (I) Acetate in Hops Toothpaste (Aged in Vial)

When making the copper (I) acetate stock solution, a precipitate formed that stayed at the bottom of the flask due to its poor water solubility. Based on this observation, copper (I) acetate is added directly in solid powder form as Experiment 15A-15D. In this Experiment, the effect of copper (I) acetate on methyl mercaptan production in a hops toothpaste was evaluated. TABLE 34 describes a control Example 15A and Examples 15B-15D containing copper (I) acetate. The Toothpaste Base (25 g) was combined with increasing levels of copper (I) acetate ranging from 0 ppm (control) to 212 ppm for a total of 4 test samples. The desired amount of copper (I) acetate was added to the Toothpaste Base (25 g), followed by vigorous stirring with spatula spoon for 2 minutes to ensure the evenly mixing.

TABLE 34
Toothpaste Compositions

| | | Copper (I) | | |
|---|---|---|---|---|
| | Toothpaste | acetate | Copper (I) | Cu metal |
| Example | Base (g) | Solid (mg) | Acetate (ppm) | (ppm) |
| 15A | 25.0 | 0 | 0 | 0 |
| 15B | 25.0 | 1.4 | 56 | 29 |
| 15C | 25.0 | 3.2 | 128 | 66 |
| 15D | 25.0 | 5.3 | 212 | 110 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 35, demonstrate the reduction and elimination of methyl mercaptan from Examples 15B-15D, which has been compared to and normalized against the control sample (Ex. 15A) without copper (I) acetate. This example demonstrates that some metal salts, without adequate solubility, still can be applied effectively to reduce methyl mercaptan, but it requires special handling and mixing.

TABLE 35
Impact of Copper (I) Acetate Solid on
Hops Toothpaste in a Screw-Cap Vial

| | Copper (I) | | Normalized Methyl |
|---|---|---|---|
| | Acetate | Cu metal | Mercaptan |
| Example | (ppm) | (ppm) | Peak Area (%) |
| 15A | 0 | 0 | 100 |
| 15B | 56 | 29 | 10.7 |
| 15C | 128 | 66 | 5.5 |
| 15D | 212 | 110 | 4.0 |

### Experiment 16: Copper Fluoride in Hops Toothpaste (Aged in Vial)

When making the copper fluoride stock solution, a precipitate formed that stayed at the bottom of the flask due to its poor water solubility. Based on this observation, copper fluoride is added directly in solid powder form as Experiment 16A-16D. In this Experiment, the effect of copper fluoride on methyl mercaptan production in a hops toothpaste was evaluated. TABLE 36 describes a control Example 16A and Examples 16B-16D containing copper fluoride. The Toothpaste Base (25 g) was combined with increasing levels of copper fluoride ranging from 0 ppm (control) to 200 ppm for a total of 4 test samples. The desired amount of copper fluoride was added to the Toothpaste Base (25 g), followed by vigorous stirring with spatula spoon for 2 minutes to ensure the evenly mixing.

TABLE 36
Toothpaste Compositions

| | | Copper | Copper | |
|---|---|---|---|---|
| | Toothpaste | Fluoride | Fluoride | Cu metal |
| Example | Base (g) | Solid (mg) | (ppm) | (ppm) |
| 16A | 25.0 | 0 | 0 | 0 |
| 16B | 25.0 | 1.3 | 52 | 33 |
| 16C | 25.0 | 3.0 | 120 | 75 |
| 16D | 25.0 | 5.0 | 200 | 125 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 37, demonstrate the reduction and elimination of methyl mercaptan from Examples 16B-16D, which has been compared to and normalized against the control sample (Ex. 16A) without copper fluoride. This example demonstrates that some metal salts, without adequate solubility, still can be applied effectively to reduce methyl mercaptan, but it requires special handling and mixing.

TABLE 37
Impact of Copper Fluoride Solid on
Hops Toothpaste in a Screw-Cap Vial

| | Copper | | Normalized Methyl |
|---|---|---|---|
| | Fluoride | Cu metal | Mercaptan |
| Example | (ppm) | (ppm) | Peak Area (%) |
| 16A | 0 | 0 | 100 |
| 16B | 52 | 33 | 12.3 |
| 16C | 120 | 75 | 8.4 |
| 16D | 200 | 125 | 6.4 |

### Experiment 17: Copper 0 in Hops Toothpaste (Aged in Vial)

In this Experiment 17A-17D, the effect of copper 0 metal (added in 150 mesh powder form) on methyl mercaptan production in a hops toothpaste was evaluated. TABLE 38 describes a control Example 17A and Examples 17B-17D containing copper 0 metal. The Toothpaste Base (25 g) was combined with increasing levels of copper 0 metal ranging from 0 ppm (control) to 4004 ppm for a total of 4 test samples. The desired amount of copper 0 metal was added to the Toothpaste Base (25 g), followed by vigorous stirring with spatula spoon for 2 minutes to ensure the evenly mixing.

TABLE 38
Toothpaste Compositions

| | | Copper 0 | Copper 0 | |
|---|---|---|---|---|
| | Toothpaste | metal | metal | Cu metal |
| Example | Base (g) | (mg) | (ppm) | (ppm) |
| 17A | 25.0 | 0 | 0 | 0 |
| 17B | 25.0 | 25.1 | 1004 | 1004 |
| 17C | 25.0 | 50.1 | 2004 | 2004 |
| 17D | 25.0 | 100.1 | 4004 | 4004 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 39, demonstrate the reduction and elimination of methyl mercaptan from Examples 17B-17D, which has been compared to and normalized against the control sample (Ex. 17A) without copper 0 metal. This example demonstrates that some metals like copper, still can be applied effectively to reduce methyl mercaptan.

TABLE 39
Impact of Copper 0 metal Solid on
Hops Toothpaste in a Screw-Cap Vial

| | Copper 0 | | Normalized Methyl |
|---|---|---|---|
| | Metal | Cu Metal | Mercaptan |
| Example | (ppm) | (ppm) | Peak Area (%) |
| 17A | 0 | 0 | 100 |
| 17B | 1004 | 1004 | 26.5 |
| 17C | 2004 | 2004 | 17.8 |
| 17D | 4004 | 4004 | 12.2 |

### Experiment 18: Silver (II) Oxide in Hops Toothpaste (Aged in Vial)

When making the silver (II) oxide stock solution, a precipitate formed that stayed at the bottom of the flask due to its poor water solubility. Based on this observation, silver (II) oxide is added directly in solid powder form as Experiment 18A-18D. In this Experiment, the effect of silver (II) oxide on methyl mercaptan production in a hops toothpaste was evaluated. TABLE 40 describes a control Example 18A and Examples 18B-18D containing silver (II) oxide. The Toothpaste Base (25 g) was combined with increasing levels of silver (II) oxide ranging from 0 ppm (control) to 200 ppm for a total of 4 test samples. The desired amount of silver (II) oxide was added to the Toothpaste Base (25 g), followed by vigorous stirring with spatula spoon for 2 minutes to ensure the evenly mixing.

TABLE 40
Toothpaste Compositions

| | | Silver (II) | | |
|---|---|---|---|---|
| | Toothpaste | Oxide | Silver (II) | Ag metal |
| Example | Base (g) | Solid (mg) | Oxide (ppm) | (ppm) |
| 18A | 25.0 | 0 | 0 | 0 |
| 18B | 25.0 | 1.3 | 52 | 45 |
| 18C | 25.0 | 3.0 | 120 | 105 |
| 18D | 25.0 | 5.1 | 204 | 178 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 41, demonstrate the reduction and elimination of methyl mercaptan from Examples 18B-18D, which has been compared to and normalized against the control sample (Ex. 18A) without silver (II) oxide. This example demonstrates that some metal salts, without adequate solubility, still can be applied effectively to reduce methyl mercaptan, but it requires special handling and mixing.

TABLE 41
Impact of Silver (II) Oxide Solid on
Hops Toothpaste in a Screw-Cap Vial

| | Silver (II) | | Normalized Methyl |
|---|---|---|---|
| | oxide | Ag metal | Mercaptan |
| Example | (ppm) | (ppm) | Peak Area (%) |
| 18A | 0 | 0 | 100 |
| 18B | 52 | 45 | 11.1 |
| 18C | 120 | 105 | 7.7 |
| 18D | 204 | 178 | 3.5 |

### Experiment 19: Silver (I) Oxide in Hops Toothpaste (Aged in Vial)

When making the silver (I) oxide stock solution, a precipitate formed that stayed at the bottom of the flask due to its poor water solubility. Based on this observation, silver (I) oxide is added directly in solid powder form as Experiment 19A-19D. In this Experiment, the effect of silver (I) oxide on methyl mercaptan production in a hops toothpaste was evaluated. TABLE 42 describes a control Example 19A and Examples 19B-19D containing silver (I) oxide. The Toothpaste Base (25 g) was combined with increasing levels of silver (I) oxide ranging from 0 ppm (control) to 204 ppm for a total of 4 test samples. The desired amount of silver (I) oxide was added to the Toothpaste Base (25 g), followed by vigorous stirring with spatula spoon for 2 minutes to ensure the evenly mixing.

TABLE 42
Toothpaste Compositions

| | | Silver (I) | | |
|---|---|---|---|---|
| | Toothpaste | Oxide | Silver (I) | Ag metal |
| Example | Base (g) | Solid (mg) | Oxide (ppm) | (ppm) |
| 19A | 25.0 | 0 | 0 | 0 |
| 19B | 25.0 | 1.3 | 52 | 48 |
| 19C | 25.0 | 2.6 | 104 | 97 |
| 19D | 25.0 | 5.1 | 204 | 190 |

After mixing, 3 g of each sample was weighed into a 20 mL headspace sampling vial. Before measuring the release of methyl mercaptan, the samples were aged at 50° C. for two hours. When the samples were ready, the GC/MS Testing Protocol was used to measure the amount of methyl mercaptan in each sample's headspace. The results, based on a normalized peak area, as shown in TABLE 43, demonstrate the reduction and elimination of methyl mercaptan from Examples 19B-19D, which has been compared to and normalized against the control sample (Ex. 19A) without silver (I) oxide. This example demonstrates that some metal salts, without adequate solubility, still can be applied effectively to reduce methyl mercaptan, but it requires special handling and mixing.

TABLE 43
Impact of Silver (I) Oxide Solid on
Hops Toothpaste in a Screw-Cap Vial

| | Silver (I) | | Normalized Methyl |
|---|---|---|---|
| | oxide | Ag metal | Mercaptan |
| Example | (ppm) | (ppm) | Peak Area (%) |
| 19A | 0 | 0 | 100 |
| 19B | 52 | 48 | 38.6 |
| 19C | 104 | 97 | 26.0 |
| 19D | 204 | 190 | 8.8 |

Preliminary Sensory Evaluation on Toothpaste with Salt

A wide array of copper, zinc and silver salts are commercially available and may work to varying degrees in an odor reduction oral health application. As a preliminary step, the taste impact was tested in the hops extract and stannous containing toothpaste base. TABLES 44 and 45 show the impact that each had on the overall color and taste relative to the control toothpaste base composition of TABLE 2. The same procedure and aging process was followed to prepare the sensory evaluation samples as those used to generate the analytical samples. Namely, to expedite the reaction from dimethyl disulfide to methyl mercaptan, the samples were aged in a hot box manufactured by Thermo Scientific at 50° C. for 2 hours. There was no level of copper chlorophyllin complex that reduced MM sufficiently without imparting an unacceptable taste or color. There was no level of copper(0) powder that worked without imparting an unacceptable appearance (muddy brown color).

TABLE 44
Impact of each compound on the taste and overall
color of the basic, unflavored plain toothpaste.

| | | Color Impact to |
|---|---|---|
| Compound (5 ppm) | Sensory Description vs. Control | Toothpaste Base |
| Copper Gluconate | No off-note on taste or aroma; reduced astringency | No change |
| Sodium Copper | No off-note on taste or aroma; reduced astringency | Light green |
| Chlorophyllin | | |
| Copper Sulfate | No off-note on taste or aroma | No change |
| Copper Citrate | No off-note on taste or aroma; mouth water like; | No change |
| | slight salty | |
| Copper Lactate | No off-note on taste or aroma; slight creamy | No change |
| | mouthfeel | |
| Copper (II) Acetate | No off-note on taste or aroma | No change |
| Copper (I) Acetate | No off-note on taste or aroma | No change |
| Copper and Zinc | No off-note on taste or aroma | No change |
| Gluconate Blend | | |
| Zinc Gluconate | No off-note on taste or aroma | No change |
| Zinc Oxide | No off-note on taste or aroma; reduced astringency; | No change |
| | slight sweat | |
| Zinc Lactate | No off-note on taste or aroma; slight creamy | No change |
| Zinc Citrate | No off-note on taste or aroma | No change |
| Silver Nitrate | No off-note on taste or aroma | Light brown |

TABLE 45
Dosage impact of each compound on the taste and overall
color of the basic unflavored plain toothpaste.

| | Dosage Level | |
|---|---|---|
| Compound | (ppm) | Sensory Description & Color Impact vs. Control |
| Copper Gluconate | 20 | Slight astringency; slight metallic, ACCEPTABLE |
| | 100 | Metallic; slight sour, UNACCEPTABLE |
| | 200 | Metallic; sucking on a penny, UNACCEPTABLE |
| Sodium Copper | 20 | Slight off-note, ACCEPTABLE |
| Chlorophyllin | 100 | Enhanced hops flavor and bitterness; green color, |
| | | UNACCEPTABLE |
| | 200 | Astringency; increased hops flavor; metallic; darker |
| | | green, UNACCEPTABLE |

### Discussion

Hops extracts, both refined and crude, represent an interesting and valuable addition to oral care products if their sulfur off-notes can be controlled. In the present invention, we describe a means of reducing and controlling the buildup of thiols, like, but not limited to methyl mercaptan. In this experiment, a reduction of at least 60% of the peak area of MM was needed to improve the taste of the hops-containing toothpaste base. A person skilled in the art would recognize that there are many different thiols that could be formed and that the present invention would be capable of reducing those as well. In addition to copper, we also demonstrate that other metal salts can work to reduce thiols, such as MM, including salts of silver and zinc. Of these latter metals, silver works very well, while zinc displays marginal efficacy. We have also conceived of and reduced to practice metal salt blends like, but not limited to, a blend of zinc and copper salts. While copper has previously been mentioned as a means of controlling thiols (mercaptans) in a toothpaste application containing stannous fluoride, it was not explored and, as such, the limitations of copper salts for thiol formation were not understood. In the present work, the inventors demonstrate that not all copper salts function equally. For instance, while copper gluconate and copper sulfate work well, sodium copper chlorophyllin generates an off color in application rendering it more challenging to work with when the toothpaste color is intended to be white. Copper citrate lacks good water solubility making it unsuitable for use in an aqueous toothpaste or water-based mouthwash. Whereas blends of soluble copper and zinc worked better than either alone. The collected results of the experiments are summarized in TABLE 46, which indicates what metals are able to achieve the minimal amount of MM reduction by normalized MM peak area (%) of at least 60%. In some cases, an unacceptable amount of material was needed to reach this threshold, including but not limited to Ex. 17B, 17C, 17D, Ex. 3D, Ex. 1H, Ex. 1I, Ex. 1J, Ex. 1K, Ex. 3D. In some cases, a material was not demonstrated as capable of reaching this threshold, including but not limited to zinc oxide (Ex. 10), zinc lactate (Ex. 11), zinc citrate (Ex. 12), or copper citrate pre-dissolved in water (Ex. 5). In some cases, there is a minimal amount of salt to be effective and acceptable, including but not limited to copper gluconate (effective level, Ex. 1D, vs. ineffective level, Ex. 1C) and copper lactate (effective level, Ex. 13C, vs. ineffective level, Ex. 13B). Based on these results, we have marked those examples that are preferred, via bold text and asterisk, (i.e., reduce the MM peak area to 40%, or less, of the control peak area) and use sufficiently small enough amounts of metal to not impart unacceptable taste or color (i.e., less than about 20, less than about 15, or even less than about 10 ppm metal).

TABLE 46
Comparison of Metal/Level Effect on Methyl Mercaptan Generation

| | | | Normalized Methyl |
|---|---|---|---|
| | | Metal | Mercaptan |
| Example | Metal Source | (ppm) | Peak Area (%) |
| 7B | Zinc Gluconate | 0.7 | 100.0 |
| 5H | Copper Citrate dispersed in Water | NA | 98.3 |
| 5G | Copper Citrate dispersed in Water | NA | 98.2 |
| 11D | Zinc Lactate | 53.3 | 97.9 |
| 5F | Copper Citrate dispersed in Water | NA | 97.7 |
| 12C | Zinc Citrate | 35.5 | 96.9 |
| 12D | Zinc Citrate | 69.7 | 96.6 |
| 11B | Zinc Lactate | 12.8 | 96.5 |
| 11C | Zinc Lactate | 27.7 | 96.0 |
| 12B | Zinc Citrate | 16.4 | 95.5 |
| 10B | Zinc Oxide | 41.8 | 95.3 |
| 10C | Zinc Oxide | 83.6 | 92.4 |
| 10D | Zinc Oxide | 170.3 | 91.5 |
| 7C | Zinc Gluconate | 1.4 | 91.2 |
| 3B | Sodium Copper Chlorophyllin | 3.5 | 86.6 |
| 7D | Zinc Gluconate | 2.9 | 78.1 |
| 3C | Sodium Copper Chlorophyllin | 35.4 | 64.8 |
| 1B | Copper Gluconate | 0.7 | 63.2 |
| 8B | Zinc Gluconate and Copper Gluconate | 0.8 | 51.8 |
| 1C | Copper Gluconate | 1.4 | 47.1 |
| 13B | Copper Lactate | 1.3 | 45.6 |
| 19B | Silver(I) Oxide | 48 | 38.6 |
| 1D* | Copper Gluconate | 2.8 | 37.2 |
| 8C* | Zinc Gluconate and Copper Gluconate | 1.4 | 34.9 |
| 13C* | Copper Lactate | 2.6 | 34.2 |
| 1E* | Copper Gluconate | 5.6 | 31.8 |
| 13D* | Copper Lactate | 5.3 | 27.8 |
| 17B | Copper(0) Powder | 1004 | 26.5 |
| 19C | Silver(I) Oxide | 97 | 26.0 |
| 9B* | Copper Gluconate/Silver Nitrate Blend | 2 | 24.6 |
| 2B* | Copper Gluconate | 0.7 | 24.5 |
| 1F* | Copper Gluconate | 11.3 | 24.2 |
| 6B* | Silver Nitrate | 3.2 | 20.4 |
| 8D* | Zinc Gluconate and Copper Gluconate | 2.8 | 19.9 |
| 17C | Copper(0) Powder | 2004 | 17.8 |
| 14B* | Copper(II) Acetate | 18 | 13.9 |
| 16B | Copper Fluoride | 33 | 12.3 |
| 17D | Copper(0) Powder | 4004 | 12.2 |
| 5B* | Copper Citrate in Toothpaste | 18.9 | 12.1 |
| 6C* | Silver Nitrate | 6.4 | 11.7 |
| 18B | Silver(II) Oxide | 45 | 11.1 |
| 15B | Copper(I) Acetate | 29 | 10.7 |
| 1G | Copper Gluconate | 56.4 | 10.5 |
| 9C* | Copper Gluconate/Silver Nitrate Blend | 3.9 | 10.2 |
| 19D | Silver(I) Oxide | 190 | 8.8 |
| 14C | Copper(II) Acetate | 43 | 8.7 |
| 16C | Copper Fluoride | 75 | 8.4 |
| 18C | Silver(II) Oxide | 105 | 7.7 |
| 5C | Copper Citrate in Toothpaste | 35.1 | 6.7 |
| 14D | Copper(II) Acetate | 71 | 6.5 |
| 16D | Copper Fluoride | 125 | 6.4 |
| 3D | Sodium Copper Chlorophyllin | 353.6 | 6.2 |
| 4B* | Copper Sulfate | 10.2 | 6.0 |
| 15C | Copper(I) Acetate | 66 | 5.5 |
| 5D | Copper Citrate in Toothpaste | 60.8 | 4.7 |
| 2C* | Copper Gluconate | 7 | 4.0 |
| 6D* | Silver Nitrate | 12.7 | 4.0 |
| 15D | Copper(I) Acetate | 110 | 4.0 |
| 18D | Silver(II) Oxide | 178 | 3.5 |
| 9D* | Copper Gluconate/Silver Nitrate Blend | 7.8 | 3.0 |
| 1H | Copper Gluconate | 112.8 | 2.7 |
| 2D | Copper Gluconate | 56.4 | 0.4 |
| 1I | Copper Gluconate | 281.9 | 0.0 |
| 1J | Copper Gluconate | 563.9 | 0.0 |
| 1K | Copper Gluconate | 1127.8 | 0.0 |
| 4C | Copper Sulfate | 102.4 | 0.0 |

The terms “substantially,” “essentially,” “about,” “approximately,” and the like, as may be used herein, represent the inherent degree of uncertainty that may be attributed to any quantitative comparison, value, measurement, or other representation. These terms also represent the degree by which a quantitative representation may vary from a stated reference without resulting in a change in the basic function of the subject matter at issue. Further, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
a hops extract, wherein the hops extract comprises dimethyl disulfide;
a tin ion source;
a soluble metal ion source that prevents the formation of methyl mercaptan, wherein the soluble metal ion source comprises a metal salt, a metal oxide, or a combination thereof, and the soluble metal ion source comprises a copper ion source, a silver ion source, a zinc ion source, or combinations thereof.

2. The oral care composition of claim 1, wherein the soluble metal ion source comprises a metal salt selected from copper gluconate, copper sulfate, sodium copper chlorophyllin, copper acetate, copper lactate, and combinations thereof.

3. The oral care composition of claim 1, wherein the metal salt comprises a polymer bound copper II salt.

4. The oral care composition of claim 1, wherein the soluble metal ion source comprises a zinc salt.

5. The oral care composition of claim 4, wherein the zinc salt comprises zinc gluconate.

6. The oral care composition of claim 1, further comprising a flavor extract.

7. The oral care composition of claim 6, wherein the flavor extract comprises one or more natural flavor extracts or one or more synthetic flavor extracts, or combinations thereof.

8. The oral care composition of claim 1, wherein the soluble metal ion source comprises silver ions, copper ions or a combination thereof.

9. The oral care composition of claim 1, wherein the oral care composition is a dentifrice, toothpaste or mouthrinse.

10. The oral care composition of claim 1, wherein the oral care composition comprises greater than 0 ppm to about 10,000 ppm of the soluble metal ion source.

11. The oral care composition of claim 1, further comprising fluoride.

12. The oral care composition of claim 1, wherein the oral care composition is unfermented.

13. The oral care composition of claim 1, wherein the tin ion source comprises stannous fluoride, stannous chloride, or a combination thereof.

14. The oral care composition of claim 1, wherein the oral care composition comprises greater than 0 ppm to about 200 ppm of the dimethyl disulfide.

15. A method of controlling malodor in an oral cavity, comprising:
providing an oral care composition comprising a soluble metal ion source, a tin ion source and a hops extract, wherein the metal ion source prevents the generation of or sequesters methanethiol; and
applying the oral care composition to a surface in an oral cavity of a user.

16. The method of claim 15, wherein the metal ion source comprises a copper II salt.

17. The method of claim 16, wherein the copper II salt is selected from copper gluconate, copper sulfate, sodium copper chlorophyllin, copper acetate, copper lactate, and combinations thereof.

18. The method of claim 16, wherein the copper II salt comprises a polymer bound copper II salt.
